(19) United States
(12) Patent Application Publication
(10) Pub. No.: US 2026/0253716 A1
(43) Pub. Date: Aug. 27, 2026

(54) SYSTEM

(71) Applicant: SoftBank Group Corp., Tokyo (JP)

(72) Inventor: Tomihisa TSUKAMOTO, Tokyo (JP)

(73) Assignee: SoftBank Group Corp., Tokyo (JP)

(21) Appl. No.: 19/533,297

(22) Filed: Feb. 9, 2026

(30) Foreign Application Priority Data
Feb. 21, 2025 (JP) 2025-027078

(51) Int. Cl. G16H 40/40; G16H 10/60; G16H 50/20; G16H 80/00

(52) U.S. Cl. CPC G16H 40/40; G16H 10/60; G16H 50/20; G16H 80/00

(57) ABSTRACT

The system according to the embodiment comprises a collection unit, an analysis unit, and a provision unit. The collection unit collects personal medical information. The analysis unit analyzes medical data collected by the collection unit. The provision unit provides a diagnostic result obtained by the analysis unit.

### CROSS-REFERENCE TO RELATED APPLICATION

The present application claims priority to and incorporates by reference the entire contents of Japanese Patent Application No. 2025-027078 filed in Japan on Feb. 21, 2025.

### BACKGROUND OF THE INVENTION

### 1. Field of the Invention

The technology of this disclosure relates to a system.

### 2. Description of the Related Art

Japanese Patent Application Laid-open No. 2022-180282 discloses a persona chatbot control method executed by at least one processor, comprising: receiving a user utterance, adding the user utterance to a prompt containing instructions related to the character of the chatbot, encoding the prompt, inputting the encoded prompt into a language model, and generating a chatbot utterance in response to the user utterance.

In conventional technology, the process of effectively collecting and analyzing personal medical information and providing diagnostic results has not been sufficiently streamlined, and there is room for improvement.

### SUMMARY OF THE INVENTION

The system according to the embodiment comprises a collection unit, an analysis unit, and a provision unit. The collection unit collects personal medical information. The analysis unit analyzes medical data collected by the collection unit. The provision unit provides a diagnostic result obtained by the analysis unit.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a conceptual diagram showing an example configuration of a data processing system according to the first embodiment;

FIG. 2 is a conceptual diagram showing an example of main functions of a data processing device and a smart device according to the first embodiment;

FIG. 3 is a conceptual diagram showing an example configuration of a data processing system according to the second embodiment;

FIG. 4 is a conceptual diagram showing an example of main functions of a data processing device and smart glasses according to the second embodiment;

FIG. 5 is a conceptual diagram showing an example configuration of a data processing system according to the third embodiment;

FIG. 6 is a conceptual diagram showing an example of main functions of a data processing device and a headset-type terminal according to the third embodiment;

FIG. 7 is a conceptual diagram showing an example configuration of a data processing system according to the fourth embodiment;

FIG. 8 is a conceptual diagram showing an example of main functions of a data processing device and a robot according to the fourth embodiment;

FIG. 9 shows an emotion map where multiple emotions are mapped; and

FIG. 10 shows an emotion map where multiple emotions are mapped.

### DETAILED DESCRIPTION OF THE PREFERRED EMBODIMENTS

Hereinafter, an example of an embodiment of the system related to the technology disclosed herein will be described with reference to the attached drawings.

First, the terminology used in the following description will be explained.

In the following embodiments, a processor denoted by a reference numeral (hereinafter simply referred to as “processor”) may be a single computing device or a combination of multiple computing devices. The processor may be a single type of computing device or a combination of multiple types of computing devices. Examples of computing devices include a CPU (Central Processing Unit), GPU (Graphics Processing Unit), GPGPU (General-Purpose computing on Graphics Processing Units), APU (Accelerated Processing Unit), or TPU (Tensor Processing Unit), among others.

In the following embodiments, a RAM (Random Access Memory) denoted by a reference numeral is a memory where information is temporarily stored and used as a work memory by the processor.

In the following embodiments, a storage denoted by a reference numeral is one or more non-volatile storage devices for storing various programs and parameters. Examples of non-volatile storage devices include flash memory (SSD (Solid State Drive)), magnetic disks (e.g., hard disks), or magnetic tapes, among others.

In the following embodiments, a communication I/F (Interface) denoted by a reference numeral is an interface including a communication processor and an antenna, among others. The communication I/F manages communication between multiple computers. Examples of communication standards applicable to the communication I/F include wireless communication standards such as 5G (5th Generation Mobile Communication System), Wi-Fi (registered trademark), or Bluetooth (registered trademark), among others.

In the following embodiments, “A and/or B” means “at least one of A and B.” In other words, “A and/or B” means it may be only A, only B, or a combination of A and B. Moreover, when expressing three or more items connected by “and/or,” the same concept as “A and/or B” applies.

### First Embodiment

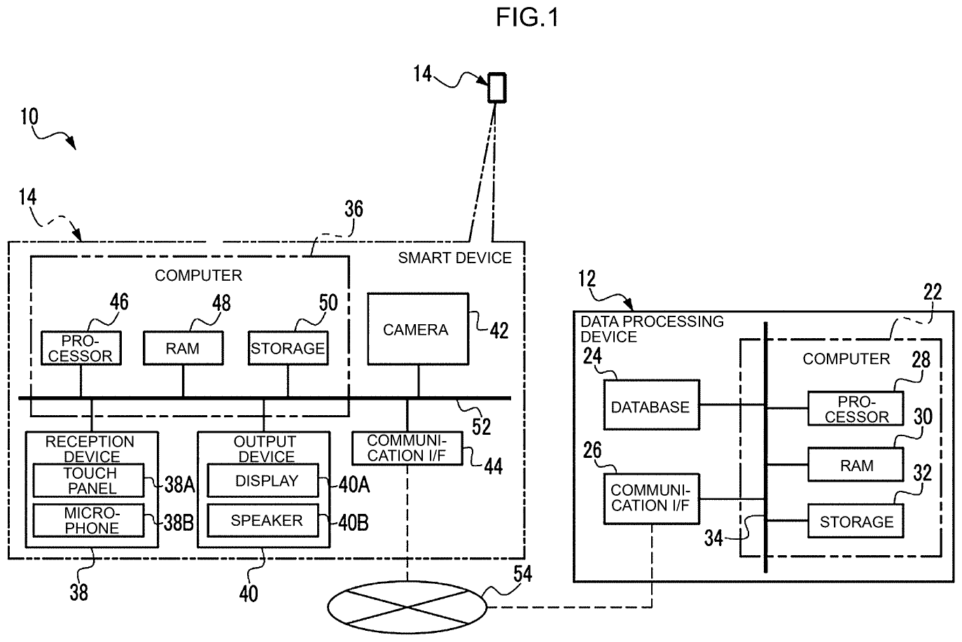

FIG. 1 shows an example configuration of a data processing system 10 according to the first embodiment.

As shown in FIG. 1, the data processing system 10 comprises a data processing device 12 and a smart device 14. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The smart device 14 comprises a computer 36, a reception device 38, an output device 40, a camera 42, and a communication I/F 44. The computer 36 comprises a processor 46, RAM 48, and storage 50. The processor 46, RAM 48, and storage 50 are connected to a bus 52. The reception device 38, output device 40, and camera 42 are also connected to the bus 52.

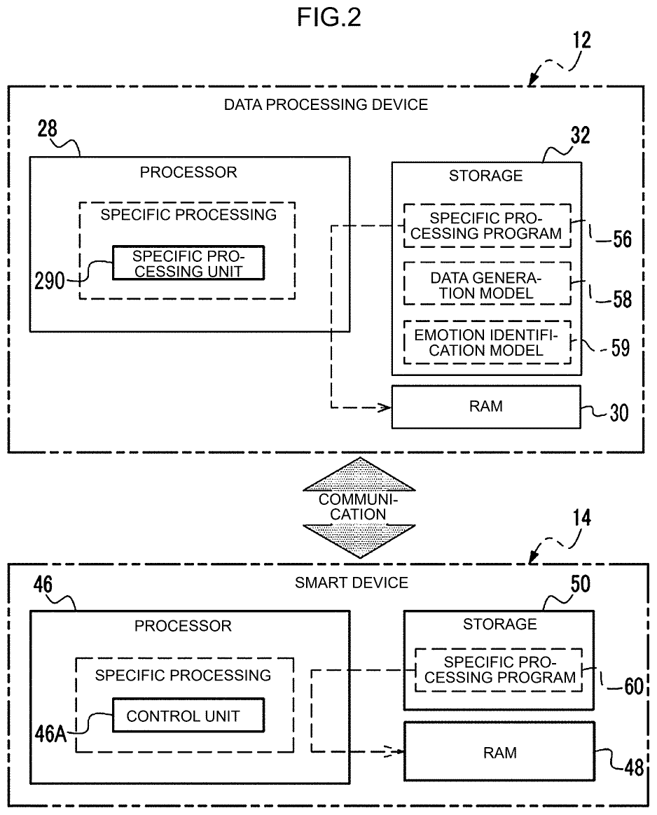

The reception device 38 comprises a touch panel 38A and a microphone 38B, among others, and accepts user input. The touch panel 38A accepts user input by detecting contact from an indicating object (e.g., a pen or finger). The microphone 38B accepts user input by detecting the user's voice. The control unit 46A sends data indicating user input accepted by the touch panel 38A and microphone 38B to the data processing device 12. The data processing device 12 has a specific processing unit 290 (see FIG. 2) that acquires data indicating user input.

The output device 40 comprises a display 40A and a speaker 40B, among others, and presents data to the user by outputting it in a perceptible form (e.g., audio and/or text). The display 40A displays visible information such as text and images according to instructions from the processor 46. The speaker 40B outputs audio according to instructions from the processor 46. The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors.

The communication I/F 44 is connected to the network 54. The communication I/F 44 and 26 manage the exchange of various information between the processor 46 and the processor 28 via the network 54.

FIG. 2 shows an example of the main functions of the data processing device 12 and the smart device 14.

As shown in FIG. 2, specific processing is performed in the data processing device 12 by the processor 28. The storage 32 stores a specific processing program 56. The specific processing program 56 is an example of a “program” related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart device 14, specific processing is performed by the processor 46. The storage 50 stores a specific processing program 60. The specific processing program 60 is used in conjunction with the specific processing program 56 by the data processing system 10. The processor 46 reads the specific processing program 60 from the storage 50 and executes it on the RAM 48. The specific processing is realized by the processor 46 operating as a control unit 46A according to the specific processing program 60 executed on the RAM 48. The smart device 14 may also have similar data generation models and emotion identification models as the data generation model 58 and emotion identification model 59, and perform the same processing as the specific processing unit 290 using these models.

Other devices besides the data processing device 12 may have the data generation model 58. For example, a server device (e.g., a generation server) may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.). Next, an example of processing by the data processing system 10 according to the first embodiment will be described.

### Example of the Embodiment

The medical information management system according to the embodiment of the present invention is a system that manages and databases personal medical information in cooperation with medical institutions. This medical information management system collects personal medical information from medical institutions and stores it in a database. Next, an AI model is developed to analyze medical images and patient data, and medical diagnostic support is provided based on the patient's past database and data from patients with similar cases. This system functions as a support feature for medical care in remote areas without large hospitals and in urban medical institutions where waiting for diagnosis takes time, and can be useful for early cancer detection and prognosis prediction of diseases. For example, personal medical information is collected from medical institutions. At this time, detailed data such as patient medical records, test results, and medical images are collected. For example, this includes patient X-ray images, MRI images, and blood test results. These data are stored in the database as personal medical information. Next, an AI model is developed to analyze medical images and patient data. The AI model analyzes the collected medical data and detects abnormalities. For example, the AI can analyze X-ray images and detect abnormalities in the lungs. In addition, the AI provides diagnostic results based on the patient's past database and data from patients with similar cases. As a result, physicians can refer to the AI's diagnostic results to make more accurate diagnoses. Furthermore, this system functions as a support feature for medical care in remote areas without large hospitals and in urban medical institutions where waiting for diagnosis takes time. For example, in remote medical institutions, patient medical data can be analyzed using the AI model and diagnostic results can be provided. This enables patients in remote areas to receive diagnoses quickly. In urban medical institutions, the AI model can be used to promptly provide diagnostic results to patients who require time to wait for diagnosis. With this system, early cancer detection and prognosis prediction of diseases become possible. For example, the AI can analyze patient medical data and detect signs of early cancer. The AI can also predict disease prognosis based on past data. As a result, patients can receive appropriate treatment at an early stage, and improvement in prognosis can be expected. Thus, the medical information management system can efficiently collect, analyze, and provide personal medical information. Specifically, this medical information management system collaborates with electronic medical record systems of multiple medical institutions, databases of testing agencies, and image management systems (such as PACS) via secure communication protocols, and automatically acquires medical records (text data, ICD codes, etc.), test results (numerical vectors, time-series data), and medical images (three-dimensional tensors in DICOM format, etc.) for each patient, assigning a unique identifier. The system normalizes and anonymizes these data and stores them in the database, thereby achieving both privacy protection and data integrity. Next, the system includes an analysis unit composed of multiple AI models, such as convolutional neural networks (CNN) for medical image analysis, recurrent neural networks (RNN) for time-series data analysis, and large language models (LLM) for medical record analysis. For example, the CNN takes a 224×224×3 X-ray image tensor as input and outputs a segmentation map of abnormal regions (binary mask) and an abnormality probability score (0.0-1.0). The RNN takes a time-series array of blood test values (e.g., numerical vectors for 30 days×20 items) as input and outputs predictions of abnormal value occurrence and disease progression risk scores. The LLM takes medical record text (e.g., “fever, cough, medical history: diabetes”) as input and outputs disease name labels and recommended treatment policy text. For training the AI models, supervised learning using past case databases (cross-entropy loss function, Adam optimization, etc.) is applied to improve model accuracy. The output of the AI models is used for subsequent processing such as threshold judgment (e.g., abnormality probability of 0.7 or higher is judged as abnormal), alert notification to physicians, automatic generation of diagnostic reports, and automatic recording in electronic medical records. Thus, this system can automatically and rapidly perform pattern extraction in high-dimensional feature space and integrated analysis of multiple data sources, unlike subjective judgment and manual data confirmation by human physicians. As a technical effect, this system achieves improved diagnostic accuracy (reduced misdiagnosis rate), shortened diagnostic time (real-time analysis), efficient data management (prevention of duplication and omission), and improved medical access in remote areas and regions with physician shortages. Specific application fields include remote image diagnosis support, rapid triage in emergency medical settings, chronic disease management, cancer screening, and prognosis-predictive personalized medicine. Furthermore, variations of the AI models such as adaptation to new diseases by transfer learning, accuracy improvement by ensemble learning, and addition of explainable AI (XAI) for evidence presentation are also possible. With these configurations, the present invention not only automates human tasks but also contributes to the improvement of computer technology itself and the resolution of issues in medical practice.

The medical information management system according to the embodiment comprises a collection unit, an analysis unit, and a provision unit. The collection unit collects personal medical information. Personal medical information includes, for example, medical records, test results, and medical images, but is not limited thereto. The collection unit, for example, acquires a patient's medical records from an electronic medical record system. The collection unit can also automatically acquire test results from testing agencies. Furthermore, the collection unit can acquire medical images from the image management system of a medical institution. For example, the collection unit acquires a patient's medical records from an electronic medical record system and stores them in a database. Test results are automatically acquired from testing agencies and stored in the database. Medical images are acquired from the image management system of a medical institution and stored in the database. The analysis unit analyzes medical data collected by the collection unit using AI. The analysis includes, for example, detection of abnormalities and diagnostic support, but is not limited thereto. For example, the analysis unit analyzes X-ray images using AI and detects abnormalities in the lungs. The analysis unit can also analyze MRI images using AI and detect abnormalities in the brain. Furthermore, the analysis unit can analyze blood test results using AI and detect abnormal values. For example, the analysis unit analyzes X-ray images using AI and detects abnormalities in the lungs. MRI images are analyzed using AI and abnormalities in the brain are detected. Blood test results are analyzed using AI and abnormal values are detected. The provision unit provides diagnostic results obtained by the analysis unit. Provision includes, for example, notification to physicians and feedback to patients, but is not limited thereto. For example, the provision unit notifies physicians of diagnostic results obtained by the analysis unit. The provision unit can also provide feedback of diagnostic results to patients. Furthermore, the provision unit can record diagnostic results in the electronic medical record system. For example, the provision unit notifies physicians of diagnostic results obtained by the analysis unit for reference in diagnosis. Diagnostic results are fed back to patients and used to determine treatment policies. Diagnostic results are recorded in the electronic medical record system and utilized for future medical care. Thus, the medical information management system according to the embodiment can efficiently collect, analyze, and provide personal medical information. Specifically, this medical information management system collaborates with electronic medical record systems of multiple medical institutions, databases of testing agencies, and image management systems via secure communication protocols (e.g., TLS/SSL), and automatically acquires medical records (text data, ICD codes, etc.), test results (numerical vectors, time-series data), and medical images (three-dimensional tensors in DICOM format, etc.) for each patient, assigning a unique identifier. The system normalizes (e.g., unit unification, missing value completion) and anonymizes (e.g., hashing of personal names and IDs) these data and stores them in the database, thereby achieving both privacy protection and data integrity. The analysis unit is composed of multiple AI models, such as convolutional neural networks (CNN) for medical image analysis, recurrent neural networks (RNN) for time-series data analysis, and large language models (LLM) for medical record analysis. For example, the CNN takes a 224×224×3 X-ray image tensor as input and outputs a segmentation map of abnormal regions (binary mask) and an abnormality probability score (0.0-1.0). The RNN takes a time-series array of blood test values (e.g., numerical vectors for 30 days×20 items) as input and outputs predictions of abnormal value occurrence and disease progression risk scores. The LLM takes medical record text (e.g., “fever, cough, medical history: diabetes”) as input and outputs disease name labels and recommended treatment policy text. For training the AI models, supervised learning using past case databases (cross-entropy loss function, Adam optimization, etc.) is applied to improve model accuracy. The output of the AI models is used for subsequent processing such as threshold judgment (e.g., abnormality probability of 0.7 or higher is judged as abnormal), alert notification to physicians, automatic generation of diagnostic reports, and automatic recording in electronic medical records. Thus, this system can automatically and rapidly perform pattern extraction in high-dimensional feature space and integrated analysis of multiple data sources, unlike subjective judgment and manual data confirmation by human physicians. As a technical effect, this system achieves improved diagnostic accuracy (reduced misdiagnosis rate), shortened diagnostic time (real-time analysis), efficient data management (prevention of duplication and omission), and improved medical access in remote areas and regions with physician shortages. Specific application fields include remote image diagnosis support, rapid triage in emergency medical settings, chronic disease management, cancer screening, and prognosis-predictive personalized medicine. Furthermore, variations of the AI models such as adaptation to new diseases by transfer learning, accuracy improvement by ensemble learning, and addition of explainable AI (XAI) for evidence presentation are also possible. With these configurations, the present invention not only automates human tasks but also contributes to the improvement of computer technology itself and the resolution of issues in medical practice.

Furthermore, the medical information management system comprises a remote support unit that supports use at remote medical institutions. The remote support unit supports use at remote medical institutions. For example, the remote support unit collects medical data from remote medical institutions and transmits it to the analysis unit. The remote support unit can also provide diagnostic results obtained from the analysis unit to remote medical institutions. For example, the remote support unit collects medical data from remote medical institutions and transmits it to the analysis unit. The analysis unit analyzes the collected medical data and obtains diagnostic results. The diagnostic results are provided to remote medical institutions via the remote support unit. Thus, diagnostic results can be promptly provided even at remote medical institutions. Some or all of the above-described processing in the remote support unit may be performed using AI or without using AI. For example, the remote support unit can input medical data collected from remote medical institutions into generative AI and have the generative AI analyze the data. Specifically, the remote support unit automatically receives medical records (text data, ICD codes, etc.), test results (numerical vectors, time-series data), and medical images (three-dimensional tensors in DICOM format, etc.) assigned with a unique identifier for each patient from remote medical institutions via secure communication protocols (e.g., TLS/SSL). The remote support unit normalizes (e.g., unit unification, missing value completion) and anonymizes (e.g., hashing of personal names and IDs) the received data and transfers it to the analysis unit. The analysis unit performs analysis using a combination of multiple AI models, such as convolutional neural networks (CNN) for medical image analysis, recurrent neural networks (RNN) for time-series data analysis, and large language models (LLM) for medical record analysis. For example, the CNN takes a 224×224×3 X-ray image tensor as input and outputs a segmentation map of abnormal regions and an abnormality probability score. The RNN takes a time-series array of blood test values (e.g., numerical vectors for 30 days×20 items) as input and outputs predictions of abnormal value occurrence and disease progression risk scores. The LLM takes medical record text (e.g., “fever, cough, medical history: diabetes”) as input and outputs disease name labels and recommended treatment policy text. For training the AI models, supervised learning using past case databases (cross-entropy loss function, Adam optimization, etc.) is applied to improve model accuracy. The output of the AI models is used for subsequent processing such as threshold judgment (e.g., abnormality probability of 0.7 or higher is judged as abnormal), alert notification to physicians, automatic generation of diagnostic reports, and automatic recording in electronic medical records. The remote support unit returns diagnostic results (e.g., binary mask images of abnormal regions, abnormality probability scores, disease name labels, recommended treatment policy text, etc.) obtained from the analysis unit to remote medical institutions in real time. The remote support unit can also be equipped with retransmission control considering communication delays and packet loss, as well as data compression and encryption functions. Furthermore, the remote support unit can use generative AI (e.g., multimodal generative AI) to integratively analyze medical images and medical records and automatically generate explainable diagnostic evidence and patient-oriented explanatory texts. Examples of AI input include DICOM image tensors (e.g., brain MRI 256×256×128), time-series vectors of blood test values (e.g., 30 days×20 items), and medical record text (e.g., “fever, cough, medical history: diabetes”). Examples of AI output include binary mask images of abnormal regions, abnormality probability scores (0.0-1.0), disease name labels (e.g., “pneumonia,” “cerebral infarction”), and recommended treatment policy text (e.g., “antibiotic administration recommended”). These outputs are used for diagnostic support by physicians at remote medical institutions, explanations to patients, automatic generation of diagnostic reports, and automatic recording in electronic medical records. As a technical effect, the remote support unit improves diagnostic accuracy at remote medical institutions, shortens diagnostic time, enhances medical access in regions with physician shortages, improves data management (prevention of duplication and omission), and protects privacy. Specific application fields include remote image diagnosis support for islands and mountainous areas, rapid triage in emergency medical settings, chronic disease management, cancer screening, and prognosis-predictive personalized medicine. Thus, the present invention not only automates human tasks but also contributes to the improvement of computer technology itself and the resolution of issues in medical practice.

Furthermore, the medical information management system comprises a rapid diagnosis unit configured to promptly provide diagnostic results to patients who require time to wait for diagnosis. The rapid diagnosis unit promptly provides diagnostic results to patients who require time to wait for diagnosis. For example, the rapid diagnosis unit collects medical data of patients who require time to wait for diagnosis and transmits it to the analysis unit. The rapid diagnosis unit can also promptly provide diagnostic results obtained from the analysis unit to patients. For example, the rapid diagnosis unit collects medical data of patients who require time to wait for diagnosis and transmits it to the analysis unit. The analysis unit analyzes the collected medical data and obtains diagnostic results. The diagnostic results are promptly provided to patients via the rapid diagnosis unit. Thus, diagnostic results can be promptly provided to patients who require time to wait for diagnosis. Some or all of the above-described processing in the rapid diagnosis unit may be performed using AI or without using AI. For example, the rapid diagnosis unit can input medical data of patients who require time to wait for diagnosis into generative AI and have the generative AI analyze the data. Specifically, the rapid diagnosis unit automatically acquires medical records (text data, ICD codes, etc.) from the electronic medical record system of patients who require time to wait for diagnosis, test results (numerical vectors, time-series data) from testing agencies, and medical images (three-dimensional tensors in DICOM format, etc.) from image management systems. The rapid diagnosis unit normalizes (e.g., unit unification, missing value completion) and anonymizes (e.g., hashing of personal names and IDs) these data and transfers them to the analysis unit in real time. The analysis unit performs analysis using a combination of multiple AI models, such as convolutional neural networks (CNN) for medical image analysis, recurrent neural networks (RNN) for time-series data analysis, and large language models (LLM) for medical record analysis. For example, the CNN takes a 224×224×3 X-ray image tensor as input and outputs a segmentation map of abnormal regions (binary mask) and an abnormality probability score (0.0-1.0). The RNN takes a time-series array of blood test values (e.g., numerical vectors for 30 days×20 items) as input and outputs predictions of abnormal value occurrence and disease progression risk scores. The LLM takes medical record text (e.g., “fever, cough, medical history: diabetes”) as input and outputs disease name labels and recommended treatment policy text. For training the AI models, supervised learning using past case databases (cross-entropy loss function, Adam optimization, etc.) is applied to improve model accuracy. The output of the AI models is used for subsequent processing such as threshold judgment (e.g., abnormality probability of 0.7 or higher is judged as abnormal), alert notification to physicians, automatic generation of diagnostic reports, and automatic recording in electronic medical records. The rapid diagnosis unit returns diagnostic results (e.g., binary mask images of abnormal regions, abnormality probability scores, disease name labels, recommended treatment policy text, etc.) obtained from the analysis unit to patients and physicians in real time. The rapid diagnosis unit can also be equipped with retransmission control considering communication delays and packet loss, as well as data compression and encryption functions. Furthermore, the rapid diagnosis unit can use generative AI (e.g., multimodal generative AI) to integratively analyze medical images and medical records and automatically generate explainable diagnostic evidence and patient-oriented explanatory texts. Examples of AI input include DICOM image tensors (e.g., brain MRI 256×256×128), time-series vectors of blood test values (e.g., 30 days×20 items), and medical record text (e.g., “fever, cough, medical history: diabetes”). Examples of AI output include binary mask images of abnormal regions, abnormality probability scores (0.0-1.0), disease name labels (e.g., “pneumonia,” “cerebral infarction”), and recommended treatment policy text (e.g., “antibiotic administration recommended”). These outputs are used for diagnostic support by patients and physicians, explanations to patients, automatic generation of diagnostic reports, and automatic recording in electronic medical records. As a technical effect, the rapid diagnosis unit improves diagnostic accuracy, shortens diagnostic time, enhances medical access in regions with physician shortages, improves data management (prevention of duplication and omission), and protects privacy for patients who require time to wait for diagnosis. Specific application fields include shortening of waiting times for diagnosis in large hospitals in urban areas, rapid triage in emergency medical settings, chronic disease management, cancer screening, and prognosis-predictive personalized medicine. Thus, the present invention not only automates human tasks but also contributes to the improvement of computer technology itself and the resolution of issues in medical practice.

The collection unit can collect data of a patient's medical records, test results, and medical images. For example, the collection unit acquires a patient's medical records from an electronic medical record system. Medical records include diagnostic results and treatment history. For example, the collection unit acquires a patient's medical records from an electronic medical record system and stores them in a database. The collection unit can also automatically acquire test results from testing agencies. Test results include blood test results and image test results. For example, the collection unit automatically acquires blood test results from testing agencies and stores them in a database. Furthermore, the collection unit can acquire medical images from the image management system of a medical institution. Medical images include X-ray images and MRI images. For example, the collection unit acquires X-ray images from the image management system of a medical institution and stores them in a database. Thus, the collection unit can collect detailed medical data of patients. Specifically, the collection unit collaborates with electronic medical record systems of multiple medical institutions, databases of testing agencies, and image management systems (such as PACS) via secure communication protocols (e.g., TLS/SSL), and automatically acquires medical records (text data, ICD codes, etc.), test results (numerical vectors, time-series data), and medical images (three-dimensional tensors in DICOM format, etc.) for each patient, assigning a unique identifier (e.g., UUID). The collection unit performs normalization processing (e.g., unit unification, missing value completion) and anonymization processing (e.g., hashing of personal names and IDs) on the acquired data and stores them in the database, thereby achieving both privacy protection and data integrity. For example, medical records such as text data “fever, cough, medical history: diabetes,” test results such as blood test value vectors for 30 days×20 items, and medical images such as 224×224×3 X-ray image tensors and 256×256×128 brain MRI tensors can be collected. These data are used as input data for high-dimensional feature extraction, abnormality detection, and disease prediction in the subsequent AI analysis unit. As a technical effect, the collection unit solves conventional issues such as manual data collection, input errors, and data duplication or omission by humans, and realizes real-time and highly accurate medical data collection. As a result, effects such as improved diagnostic accuracy, shortened diagnostic time, efficient data management, and improved medical access in remote areas and regions with physician shortages can be obtained in medical practice. Specific application fields include remote image diagnosis support, rapid triage in emergency medical settings, chronic disease management, cancer screening, and prognosis-predictive personalized medicine. Furthermore, the collection unit can flexibly operate by selecting data acquisition methods such as batch acquisition, streaming acquisition, and event-triggered acquisition according to the system configuration and network environment of medical institutions, which is also a technical advantage. With these configurations, the present invention not only automates human tasks but also contributes to the improvement of computer technology itself and the resolution of issues in medical practice.

The analysis unit can analyze medical data using AI and detect abnormalities. For example, the analysis unit analyzes X-ray images using AI and detects abnormalities in the lungs. The AI receives X-ray images as input and executes algorithms to detect abnormalities. For example, the AI identifies abnormal regions in X-ray images and highlights those regions. The analysis unit can also analyze MRI images using AI and detect abnormalities in the brain. The AI receives MRI images as input and executes algorithms to detect abnormalities. For example, the AI identifies abnormal regions in MRI images and highlights those regions. Furthermore, the analysis unit can analyze blood test results using AI and detect abnormal values. The AI receives blood test results as input and executes algorithms to detect abnormal values. For example, the AI identifies values that deviate from normal ranges in blood test results and highlights those values. Thus, the analysis unit can improve the accuracy of medical data analysis by using AI. Specifically, the analysis unit is composed of multiple AI models, such as convolutional neural networks (CNN) for medical image analysis, recurrent neural networks (RNN) for time-series data analysis, and large language models (LLM) for medical record analysis. The CNN takes 224×224×3 X-ray image tensors and 256×256×128 MRI image tensors as input and outputs segmentation maps of abnormal regions (binary mask images) and abnormality probability scores (0.0-1.0). The RNN takes time-series arrays of blood test values (e.g., numerical vectors for 30 days×20 items) as input and outputs predictions of abnormal value occurrence and disease progression risk scores. The LLM takes medical record text (e.g., “fever, cough, medical history: diabetes”) as input and outputs disease name labels and recommended treatment policy text. For training the AI models, supervised learning using past case databases (cross-entropy loss function, Adam optimization, etc.) is applied to improve model accuracy. The output of the AI models is used for subsequent processing such as threshold judgment (e.g., abnormality probability of 0.7 or higher is judged as abnormal), alert notification to physicians, automatic generation of diagnostic reports, and automatic recording in electronic medical records. For example, CNN output examples include binary mask images indicating abnormal regions in the lung field and scores such as “abnormality probability 0.85.” RNN output examples include “increased risk of anemia within 7 days” and “disease progression risk 0.65.” LLM output examples include text such as “disease name: pneumonia” and “recommended treatment: antibiotic administration.” These outputs are used for subsequent provision units, diagnostic support by physicians, patient explanations, and automatic generation of diagnostic reports. As a technical effect, the analysis unit can automatically and rapidly perform pattern extraction in high-dimensional feature space and integrated analysis of multiple data sources, unlike subjective judgment and manual data confirmation by human physicians, thereby improving diagnostic accuracy (reducing misdiagnosis rate), shortening diagnostic time (real-time analysis), and improving data management (prevention of duplication and omission). Specific application fields include remote image diagnosis support, rapid triage in emergency medical settings, chronic disease management, cancer screening, and prognosis-predictive personalized medicine. Furthermore, variations of the AI models such as adaptation to new diseases by transfer learning, accuracy improvement by ensemble learning, and addition of explainable AI (XAI) for evidence presentation are also possible. With these configurations, the present invention not only automates human tasks but also contributes to the improvement of computer technology itself and the resolution of issues in medical practice.

The provision unit can provide diagnostic results analyzed by AI to physicians. For example, the provision unit notifies physicians of diagnostic results analyzed by AI. The AI receives analysis results as input and generates diagnostic results. For example, the AI diagnoses lung abnormalities based on analysis results of X-ray images. The provision unit can also provide feedback of diagnostic results to patients. The AI receives analysis results as input and generates diagnostic results. For example, the AI diagnoses brain abnormalities based on analysis results of MRI images. Furthermore, the provision unit can record diagnostic results in the electronic medical record system. The AI receives analysis results as input and generates diagnostic results. For example, the AI diagnoses abnormal values based on analysis results of blood test results. Thus, the provision unit can improve diagnostic accuracy by providing diagnostic results analyzed by AI to physicians. Specifically, the provision unit is equipped with functions to automatically distribute and record diagnostic results output from the analysis unit (e.g., binary mask images of abnormal regions, abnormality probability scores, disease name labels, recommended treatment policy text, etc.) to various output destinations such as physicians, patients, and electronic medical record systems. The provision unit can dynamically switch notification methods (e.g., alerts to physicians, explanatory texts for patients, automatic generation of diagnostic reports, automatic recording in electronic medical records, etc.) and level of detail (e.g., summary, detailed explanation) according to the content and importance of diagnostic results. For example, detailed analysis results such as “abnormality probability 0.85, abnormal region in lower right lung field” can be notified to physicians, and easy-to-understand explanatory texts such as “An abnormality was found in your lung. The physician will provide a detailed explanation” can be provided to patients. Structured data of diagnostic results (e.g., coordinates of abnormal regions, abnormality scores, recommended treatment policy, etc.) can be automatically recorded in electronic medical record systems for use in future medical care and statistical analysis. Furthermore, the provision unit supports multiple output formats for diagnostic results, such as images (e.g., mask images highlighting abnormal regions), numerical values (e.g., abnormality probability scores), and text (e.g., disease name, treatment policy), and can select the optimal format according to the output destination and purpose of use. As a technical effect, the provision unit prevents transmission errors and information omissions of diagnostic results, supports physician decision-making, improves efficiency of patient explanations, reduces workload through automation of medical record keeping, and improves diagnostic accuracy. Specific application fields include remote diagnosis support, rapid triage in emergency medical settings, chronic disease management, cancer screening, and prognosis-predictive personalized medicine. Furthermore, the provision unit can dynamically control the output timing and priority of diagnostic results according to the situation in medical practice and the urgency of patients, thereby realizing highly real-time and flexible diagnostic support. With these configurations, the present invention not only automates human tasks but also contributes to the improvement of computer technology itself and the resolution of issues in medical practice.

The collection unit can estimate a patient's emotion and adjust the timing of medical data collection based on the estimated emotion of the patient. For example, if the patient is feeling anxious, the collection unit collects medical data at a time when the patient can relax. Emotion estimation is realized using an emotion engine or generative AI, such as emotion estimation functions. Generative AI may include text generation AI (e.g., LLM) or multimodal generative AI, but is not limited thereto. For example, the collection unit captures the patient's facial expressions with a camera and estimates emotion using an emotion estimation algorithm. The emotion score is calculated based on changes in facial expressions, and the collection timing is adjusted. The collection unit can also record the patient's voice and estimate emotion using voice analysis technology. The tone and speed of the voice are analyzed, the emotion score is calculated, and the collection timing is adjusted. Furthermore, the collection unit can collect the patient's biometric data (heart rate and skin electrical activity) with sensors and estimate emotion using an emotion estimation algorithm. The emotion score is calculated based on heart rate variability, and the collection timing is adjusted. Thus, by adjusting the timing of medical data collection according to the patient's emotion, data can be collected at a more appropriate timing. Specifically, the collection unit simultaneously acquires multiple sensor data for emotion estimation of the patient (e.g., facial image 224×224×3 RGB tensor, one-dimensional time-series array of voice waveform, numerical vector of heart rate and skin electrical activity, etc.). The collection unit preprocesses these data (e.g., face region extraction from facial images, noise removal from voice, normalization of vital data) and inputs them to an AI model for emotion estimation. The AI model may use a convolutional neural network (CNN) for facial images, a recurrent neural network (RNN) or a model with self-attention mechanism for voice, and a multilayer perceptron (MLP) for biometric data. For example, the CNN takes a facial image tensor as input and outputs a probability distribution of emotion labels such as “joy,” “anxiety,” and “anger” (e.g., anxiety 0.75, relaxation 0.15, joy 0.10). The RNN takes a voice waveform as input and outputs an emotion score (e.g., anxiety level 0.68) from the tone and speed of the voice. The MLP takes a time-series vector of heart rate and skin electrical activity as input and outputs scores for stress level and relaxation level (e.g., stress 0.80). The collection unit integrates the outputs of these multiple AI models and calculates the final emotion estimation result (e.g., anxiety level 0.72). The emotion estimation result is input to the collection timing control module, and the timing for medical data collection is automatically determined by rule-based or machine learning-based timing optimization algorithms, such as “delay collection by 10 minutes if anxiety level is 0.7 or higher” or “collect immediately if relaxation level is 0.8 or higher.” For training the AI models, supervised learning using multimodal datasets with emotion labels (cross-entropy loss function, Adam optimization, etc.) is applied to improve estimation accuracy. As a technical effect, the collection unit can integratively analyze multiple biometric and behavioral data in high-dimensional feature space without relying on subjective judgment or empirical rules of human operators, thereby minimizing psychological burden on patients and collecting high-quality medical data at optimal timing. This reduces vital fluctuations and noise contamination caused by stress during data collection, contributing to improved accuracy of medical AI analysis and increased patient satisfaction. Specific application fields include medical data collection for children and the elderly, vital monitoring for patients with mental disorders, stress-free data acquisition in remote medical settings, and reduction of subject burden in clinical trials and research. Furthermore, variations of the emotion estimation AI model such as adaptation to individual characteristics by transfer learning, improvement of estimation accuracy by ensemble learning, and addition of explainable AI (XAI) for presenting the basis of emotion estimation are also possible. With these configurations, the present invention not only automates human tasks but also contributes to the improvement of computer technology itself and the resolution of issues in medical practice.

The collection unit can analyze a patient's past medical records and select an optimal collection method. For example, the collection unit selects the most effective collection method from the patient's past medical records. Collection methods include, for example, data collection means and collection frequency, but are not limited thereto. For example, the collection unit analyzes the patient's past medical records and determines the types of data to be collected. Past medical records include diagnostic results and treatment history. For example, the collection unit determines the types of data to be collected based on the patient's past medical records. The collection unit can also analyze the patient's past medical records and optimize the collection frequency. For example, the collection unit optimizes the collection frequency based on the patient's past medical records. Thus, by selecting the optimal collection method based on the patient's past medical records, the collection unit can efficiently collect data. Specifically, the collection unit refers to a medical record database assigned with a unique identifier for each patient (e.g., diagnostic results, treatment history, medication history, time-series vectors of test values, etc.) and uses machine learning algorithms (e.g., decision trees, random forests, reinforcement learning, etc.) to automatically optimize collection means (e.g., wearable sensors, regular interviews, remote monitoring, etc.) and collection frequency (e.g., once a day, once a week, event-triggered, etc.). The collection unit extracts features of medical records (e.g., frequency of past disease onset, treatment responsiveness, variation patterns of test values, etc.) and inputs these as input vectors (e.g., time-series data for 30 items over the past year) to the AI model. The AI model outputs structured data such as “recommended collection means label (e.g., wearable sensor),” “recommended collection frequency (e.g., twice a day),” and “recommended data types (e.g., blood glucose, heart rate, activity level).” For example, if blood glucose variation is large in the past records of a diabetes patient, high-frequency collection of blood glucose is recommended, and if there is a history of heart disease, continuous monitoring of heart rate and ECG data is recommended. For training the AI models, reinforcement learning or Bayesian optimization is applied using past collection history and the relationship between diagnostic accuracy and treatment effect as training data, aiming to optimize collection strategies. The output of the AI model is input to the control module of the collection unit, and the actual data collection process (e.g., sensor activation timing, notification timing of interview apps, etc.) is automatically controlled. Thus, the collection unit can automatically generate personalized collection strategies based on each patient's medical history, unlike empirical rules and manual collection planning by human operators. As a technical effect, the collection unit achieves reduction of communication and storage load by eliminating unnecessary data collection, prevention of missing important data, optimization of collection costs, and improvement of diagnostic and treatment accuracy. Specific application fields include chronic disease management (e.g., diabetes, heart failure), remote monitoring, data collection optimization in clinical trials and research, and home medical care support. Furthermore, variations of the AI model such as adaptation to new diseases by transfer learning, diversification of collection strategies by ensemble learning, and addition of explainable AI (XAI) for presenting the basis of collection strategies are also possible. With these configurations, the present invention not only automates human tasks but also contributes to the improvement of computer technology itself and the resolution of issues in medical practice.

The collection unit can perform filtering based on a patient's current health condition and lifestyle habits when collecting medical data. For example, the collection unit considers the patient's current health condition and filters the data to be collected. Filtering includes, for example, selection criteria for data and filtering algorithms, but is not limited thereto. For example, the collection unit determines the priority of data to be collected based on the patient's health condition. Health condition includes, for example, current disease status and treatment status. For example, the collection unit determines the priority of data to be collected based on the patient's health condition. The collection unit can also adjust the types of data to be collected based on the patient's lifestyle habits. Lifestyle habits include, for example, dietary and exercise habits. For example, the collection unit adjusts the types of data to be collected based on the patient's lifestyle habits. Thus, by filtering data according to the patient's health condition and lifestyle habits, the collection unit can collect more relevant data. Specifically, the collection unit refers to a health condition database assigned with a unique identifier for each patient (e.g., latest diagnosis, diseases under treatment, medication status, time-series vectors of vital signs, etc.) and a lifestyle habits database (e.g., dietary records, amount of exercise, sleep patterns, smoking and drinking habits, etc.), and uses filtering algorithms (e.g., rule-based, decision trees, neural networks, etc.) to automatically select and prioritize data to be collected. The collection unit inputs features of health condition and lifestyle habits (e.g., BMI, HbA1c value, daily steps, average sleep time, etc.) as input vectors (e.g., time-series data for 20 items over the past 30 days) to the AI model. The AI model outputs structured data such as “recommended data types (e.g., blood glucose, blood pressure, dietary content)” and “priority scores (e.g., blood glucose 0.9, blood pressure 0.7).” For example, for patients with a tendency toward obesity and lack of exercise, high-frequency collection of dietary and exercise data is recommended, and for patients under treatment for hypertension, priority collection of blood pressure data is recommended. For training the AI models, supervised learning using past health condition and lifestyle habits and the relationship with diagnostic accuracy and treatment effect (cross-entropy loss function, Adam optimization, etc.) is applied to improve filtering accuracy. The output of the AI model is input to the control module of the collection unit, and the actual data collection process (e.g., sensor activation, interview item selection, extraction from database, etc.) is automatically controlled. Thus, the collection unit can realize personalized data collection based on each patient's health condition and lifestyle habits, unlike empirical rules and uniform collection by human operators. As a technical effect, the collection unit achieves reduction of communication and storage load by eliminating unnecessary data collection, prevention of missing important data, optimization of collection costs, and improvement of diagnostic and treatment accuracy. Specific application fields include lifestyle disease management, remote monitoring, data collection optimization in clinical trials and research, and home medical care support. Furthermore, variations of the AI model such as adaptation to new diseases by transfer learning, improvement of filtering accuracy by ensemble learning, and addition of explainable AI (XAI) for presenting the basis of filtering are also possible. With these configurations, the present invention not only automates human tasks but also contributes to the improvement of computer technology itself and the resolution of issues in medical practice.

The collection unit can estimate a patient's emotion and determine the priority of medical data to be collected based on the estimated emotion of the patient. For example, if the patient is feeling anxious, the collection unit prioritizes the collection of important data. Emotion estimation is realized using an emotion engine or generative AI, such as emotion estimation functions. Generative AI may include text generation AI (e.g., LLM) or multimodal generative AI, but is not limited thereto. For example, the collection unit captures the patient's facial expressions with a camera and estimates emotion using an emotion estimation algorithm. The emotion score is calculated based on changes in facial expressions, and the priority of data to be collected is determined. The collection unit can also record the patient's voice and estimate emotion using voice analysis technology. The tone and speed of the voice are analyzed, the emotion score is calculated, and the priority of data to be collected is determined. Furthermore, the collection unit can collect the patient's biometric data (heart rate and skin electrical activity) with sensors and estimate emotion using an emotion estimation algorithm. The emotion score is calculated based on heart rate variability, and the priority of data to be collected is determined. Thus, by determining the priority of medical data according to the patient's emotion, the collection unit can prioritize the collection of important data. Specifically, the collection unit simultaneously acquires multiple sensor data for emotion estimation of the patient (e.g., facial image 224×224×3 RGB tensor, one-dimensional time-series array of voice waveform, numerical vector of heart rate and skin electrical activity, etc.), preprocesses these data (e.g., face region extraction from facial images, noise removal from voice, normalization of vital data), and inputs them to an AI model for emotion estimation. The AI model may use a convolutional neural network (CNN) for facial images, a recurrent neural network (RNN) or a model with self-attention mechanism for voice, and a multilayer perceptron (MLP) for biometric data. For example, the CNN takes a facial image tensor as input and outputs a probability distribution of emotion labels such as “joy,” “anxiety,” and “anger” (e.g., anxiety 0.75, relaxation 0.15, joy 0.10). The RNN takes a voice waveform as input and outputs an emotion score (e.g., anxiety level 0.68) from the tone and speed of the voice. The MLP takes a time-series vector of heart rate and skin electrical activity as input and outputs scores for stress level and relaxation level (e.g., stress 0.80). The collection unit integrates the outputs of these multiple AI models and calculates the final emotion estimation result (e.g., anxiety level 0.72). The emotion estimation result is input to the collection priority determination module, and the priority of data to be collected is automatically determined by rule-based or machine learning-based priority optimization algorithms, such as “if anxiety level is 0.7 or higher, prioritize collection of vital data and urgent data,” or “if relaxation level is 0.8 or higher, collect all data as usual.” For training the AI models, supervised learning using multimodal datasets with emotion labels (cross-entropy loss function, Adam optimization, etc.) is applied to improve estimation accuracy. Examples of AI model output include “anxiety level 0.75→prioritize vital data,” “relaxation level 0.85→collect all data as usual.” These outputs are input to the control module of the collection unit, and the actual data collection process (e.g., sensor activation timing, order of interview app questions, etc.) is automatically controlled. Thus, the collection unit can integratively analyze multiple biometric and behavioral data in high-dimensional feature space without relying on subjective judgment or empirical rules of human operators, thereby minimizing psychological burden on patients and prioritizing the collection of important medical data. As a technical effect, the collection unit reduces vital fluctuations and noise contamination caused by stress during data collection, contributing to improved accuracy of medical AI analysis and increased patient satisfaction. Specific application fields include medical data collection for children and the elderly, vital monitoring for patients with mental disorders, stress-free data acquisition in remote medical settings, and reduction of subject burden in clinical trials and research. Furthermore, variations of the emotion estimation AI model such as adaptation to individual characteristics by transfer learning, improvement of estimation accuracy by ensemble learning, and addition of explainable AI (XAI) for presenting the basis of emotion estimation are also possible. With these configurations, the present invention not only automates human tasks but also contributes to the improvement of computer technology itself and the resolution of issues in medical practice.

The collection unit can preferentially collect highly relevant data by considering a patient's geographic location information when collecting medical data. For example, the collection unit preferentially collects highly relevant data based on the patient's geographic location information. Geographic location information includes, for example, GPS data and location information services, but is not limited thereto. For example, the collection unit determines the types of data to be collected based on the patient's geographic location information. Geographic location information includes, for example, the patient's residence and place of medical care. For example, the collection unit determines the types of data to be collected based on the patient's geographic location information. The collection unit can also adjust the priority of data to be collected based on the patient's geographic location information. For example, the collection unit adjusts the priority of data to be collected based on the patient's geographic location information. Thus, by considering the patient's geographic location information, the collection unit can preferentially collect highly relevant data. Specifically, the collection unit refers to a geographic location information database assigned with a unique identifier for each patient (e.g., GPS coordinates, address, place of medical care, movement history, etc.), extracts geographic features (e.g., urban, suburban, remote island, distance to medical institution, elevation, climate zone, etc.), and inputs these features as numerical vectors (e.g., latitude, longitude, elevation, distance, temperature, etc.) to the AI model to automatically determine the types and priority of data to be collected. The AI model may use random forests or neural networks. The AI model outputs structured data such as “recommended data types (e.g., body temperature and respiratory rate in infectious disease epidemic areas, oxygen saturation in mountainous areas, etc.)” and “priority scores (e.g., body temperature 0.9, blood pressure 0.6).” For example, for patients living in infectious disease epidemic areas, priority collection of fever and respiratory symptom data is recommended, and for patients in remote or medically underserved areas, high-frequency collection of remote monitoring data (e.g., vital signs, self-reported symptoms, etc.) is recommended. For training the AI models, supervised learning or reinforcement learning using past geographic location information and the relationship with diagnostic accuracy and treatment effect is applied to optimize collection strategies. The output of the AI model is input to the control module of the collection unit, and the actual data collection process (e.g., sensor activation, order of interview questions, etc.) is automatically controlled. Thus, the collection unit can realize personalized data collection according to the patient's geographic situation, unlike empirical rules and uniform collection by human operators. As a technical effect, the collection unit achieves improved diagnostic and treatment accuracy, reduction of unnecessary data collection, and optimization of communication and storage load by collecting data considering region-specific disease risks and medical access situations. Specific application fields include monitoring in infectious disease epidemic areas, medical support for remote islands and remote areas, health management during disasters, and personalized medicine according to regional characteristics. Furthermore, variations of the AI model such as adaptation to new regions by transfer learning, diversification of collection strategies by ensemble learning, and addition of explainable AI (XAI) for presenting the basis of collection are also possible. With these configurations, the present invention not only automates human tasks but also contributes to the improvement of computer technology itself and the resolution of issues in medical practice.

The collection unit can analyze a patient's social media activity when collecting medical data and collect relevant data. For example, the collection unit analyzes a patient's social media activity and collects relevant medical data. Social media activity includes, for example, analysis of post content and analysis of activity frequency, but is not limited thereto. For example, the collection unit collects health-related information based on the patient's social media activity. Social media activity includes, for example, health-related posts and comments. For example, the collection unit collects health-related information based on the patient's social media activity. The collection unit can also determine the types of data to be collected based on the patient's social media activity. For example, the collection unit determines the types of data to be collected based on the patient's social media activity. Thus, by analyzing a patient's social media activity, the collection unit can collect relevant medical data. Specifically, the collection unit refers to a social media activity database assigned with a unique identifier for each patient (e.g., post text, images, videos, posting frequency, comment history, number of likes, etc.), and uses natural language processing (NLP) models and image analysis models to extract health-related information. NLP models such as large language models (LLM) or BERT-based models are used to input post text (e.g., “I've been feeling unwell lately,” “I have a persistent headache,” etc.) and output health status labels (e.g., poor health, stress, lack of sleep, etc.) and symptom keywords (e.g., headache, fever, cough, etc.). Image analysis models such as CNN are used to extract features suggesting health status (e.g., dietary content, exercise scenes, medication status, etc.) from posted images. Examples of AI model output include “post content: headache→symptom label: headache,” “image: running→exercise habit present,” etc. These outputs are input to the control module of the collection unit, and the types and priority of data to be collected (e.g., prioritize vital data collection when poor health is posted, collect activity data when exercise habit is posted, etc.) are automatically determined. For training the AI models, supervised learning using social media datasets with health status labels (cross-entropy loss function, Adam optimization, etc.) is applied to improve extraction accuracy. Thus, the collection unit can automatically and rapidly analyze vast amounts of social media data and realize efficient and accurate medical data collection, unlike manual confirmation and information extraction by human operators. As a technical effect, the collection unit achieves health status understanding without relying on subjective patient reports, early detection of changes in lifestyle habits and symptoms, and automation and optimization of data collection. Specific application fields include lifestyle disease management, mental health monitoring, behavioral data collection in clinical trials and research, and remote medical support. Furthermore, variations of the AI model such as adaptation to new SNS by transfer learning, improvement of extraction accuracy by ensemble learning, and addition of explainable AI (XAI) for presenting the basis of extraction are also possible. With these configurations, the present invention not only automates human tasks but also contributes to the improvement of computer technology itself and the resolution of issues in medical practice.

The analysis unit can estimate a patient's emotion and adjust the expression method of analysis based on the estimated emotion of the patient. For example, if the patient is feeling anxious, the analysis unit uses a simple and easy-to-understand expression method. Emotion estimation is realized using an emotion engine or generative AI, such as emotion estimation functions. Generative AI may include text generation AI (e.g., LLM) or multimodal generative AI, but is not limited thereto. For example, the analysis unit captures the patient's facial expressions with a camera and estimates emotion using an emotion estimation algorithm. The emotion score is calculated based on changes in facial expressions, and the expression method of analysis is adjusted. The analysis unit can also record the patient's voice and estimate emotion using voice analysis technology. The tone and speed of the voice are analyzed, the emotion score is calculated, and the expression method of analysis is adjusted. Furthermore, the analysis unit can collect the patient's biometric data (heart rate and skin electrical activity) with sensors and estimate emotion using an emotion estimation algorithm. The emotion score is calculated based on heart rate variability, and the expression method of analysis is adjusted. Thus, by adjusting the expression method of analysis according to the patient's emotion, the analysis unit can provide more appropriate analysis results. Specifically, the analysis unit simultaneously acquires multiple sensor data for emotion estimation of the patient (e.g., facial image 224×224×3 RGB tensor, one-dimensional time-series array of voice waveform, numerical vector of heart rate and skin electrical activity, etc.), preprocesses these data (e.g., face region extraction from facial images, noise removal from voice, normalization of vital data), and inputs them to an AI model for emotion estimation. The analysis unit uses a convolutional neural network (CNN) for facial images, a recurrent neural network (RNN) or a model with self-attention mechanism for voice, and a multilayer perceptron (MLP) for biometric data. For example, the CNN takes a facial image tensor as input and outputs a probability distribution of emotion labels such as “joy,” “anxiety,” and “anger” (e.g., anxiety 0.75, relaxation 0.15, joy 0.10). The RNN takes a voice waveform as input and outputs an emotion score (e.g., anxiety level 0.68) from the tone and speed of the voice. The MLP takes a time-series vector of heart rate and skin electrical activity as input and outputs scores for stress level and relaxation level (e.g., stress 0.80). The analysis unit integrates the outputs of these multiple AI models and calculates the final emotion estimation result (e.g., anxiety level 0.72). The emotion estimation result is input to the analysis expression control module, and the expression method of analysis results is automatically determined by rule-based or machine learning-based expression optimization algorithms, such as “if anxiety level is 0.7 or higher, summarize analysis results and eliminate technical terms,” or “if relaxation level is 0.8 or higher, provide detailed analysis explanations.” For training the AI models, supervised learning using multimodal datasets with emotion labels (cross-entropy loss function, Adam optimization, etc.) is applied to improve estimation accuracy. Examples of AI model output include “anxiety level 0.75→summarize analysis results within two sentences,” “relaxation level 0.85→provide detailed evidence explanation.” These outputs are input to the control module of the analysis unit, and the actual analysis result generation process (e.g., generation of explanatory text by natural language generation AI, automatic replacement of technical terms, etc.) is automatically controlled. Thus, the analysis unit can integratively analyze multiple biometric and behavioral data in high-dimensional feature space without relying on subjective judgment or empirical rules of human operators, thereby minimizing psychological burden on patients and providing analysis results in the optimal expression. As a technical effect, the analysis unit contributes to improved patient understanding, reduced stress during explanation, improved accuracy of medical AI analysis, and increased patient satisfaction. Specific application fields include medical explanations for children and the elderly, consideration for patients with mental disorders, stress-free explanations in remote medical settings, and reduction of subject explanation burden in clinical trials and research. Furthermore, variations of the emotion estimation AI model such as adaptation to individual characteristics by transfer learning, improvement of estimation accuracy by ensemble learning, and addition of explainable AI (XAI) for presenting the basis of emotion estimation are also possible. With these configurations, the present invention not only automates human tasks but also contributes to the improvement of computer technology itself and the resolution of issues in medical practice.

The analysis unit can adjust the level of detail of analysis based on the importance of medical data during analysis. For example, the analysis unit performs detailed analysis for important medical data. Importance includes, for example, urgency and impact of data, but is not limited thereto. For example, the analysis unit performs detailed analysis for important medical data. The analysis unit can also perform simplified analysis for medical data of low importance. For example, the analysis unit performs simplified analysis for medical data of low importance. Furthermore, the analysis unit can adjust the level of detail of analysis according to the importance of medical data. For example, the analysis unit adjusts the level of detail of analysis according to the importance of medical data. Thus, by adjusting the level of detail of analysis according to the importance of medical data, the analysis unit can perform analysis efficiently. Specifically, the analysis unit is equipped with an importance evaluation module that automatically calculates importance scores such as urgency and clinical impact for each medical data. The analysis unit receives medical records (text data, ICD codes, etc.), test results (numerical vectors, time-series data), and medical images (three-dimensional tensors in DICOM format, etc.) as input data, and applies an importance judgment AI model (e.g., decision tree, random forest, neural network, etc.) to these data. The AI model extracts features such as “vital abnormality in emergency transport patients,” “suspected tumor in images,” and “rapid change in test values,” and outputs importance scores (e.g., 0.0-1.0). When the importance score is high, the analysis unit executes a multi-stage analysis flow by linking multiple AI models, such as detailed image segmentation by convolutional neural networks (CNN), time-series anomaly detection by recurrent neural networks (RNN), and detailed medical record analysis by large language models (LLM). For example, for a lung CT image with importance 0.9, three-dimensional CNN is used for abnormal region extraction, abnormality probability score calculation, and evidence presentation by explainable AI (XAI). On the other hand, for low-importance periodic health checkup data (e.g., importance 0.2), only simple threshold judgment and summarization are performed to save computational resources and improve analysis efficiency. Examples of AI model output include “importance 0.95→execute detailed analysis flow,” “importance 0.25→execute simplified analysis flow.” These outputs are input to the control module of the analysis unit, and branching of the analysis pipeline and resource allocation are automatically controlled. For training the AI models, supervised learning using past diagnostic results and clinical outcomes (cross-entropy loss function, Adam optimization, etc.) is applied to improve importance judgment accuracy. As a technical effect, the analysis unit achieves improved analysis speed by optimal allocation of computational resources, prevention of missing important cases, reduction of unnecessary detailed analysis and cost, and overall improvement of diagnostic accuracy. Specific application fields include triage support in emergency medical settings, early detection of abnormalities in chronic disease management, priority analysis in cancer screening, and efficient analysis under bandwidth constraints in remote medical care. Furthermore, variations of the importance judgment AI model such as adaptation to new diseases by transfer learning, improvement of judgment accuracy by ensemble learning, and addition of explainable AI (XAI) for presenting the basis of importance judgment are also possible. With these configurations, the present invention not only automates human tasks but also contributes to the improvement of computer technology itself and the resolution of issues in medical practice.

The analysis unit can apply different analysis algorithms according to the category of medical data during analysis. For example, the analysis unit applies image analysis algorithms to medical image data. Categories include, for example, by medical department or by symptom, but are not limited thereto. For example, the analysis unit applies image analysis algorithms to medical image data. The analysis unit can also apply numerical analysis algorithms to blood test data. For example, the analysis unit applies numerical analysis algorithms to blood test data. Furthermore, the analysis unit can apply text analysis algorithms to medical record data. For example, the analysis unit applies text analysis algorithms to medical record data. Thus, by applying appropriate analysis algorithms according to the category of medical data, the analysis unit can improve analysis accuracy. Specifically, the analysis unit is equipped with a data classification module that automatically determines the category of input data (e.g., medical images, test values, medical record text, vital time-series data, etc.). The analysis unit applies convolutional neural networks (CNN) or image analysis algorithms such as U-Net to medical images (e.g., DICOM format X-ray images 224×224×3, MRI images 256×256×128, etc.) to output segmentation maps of abnormal regions and abnormality probability scores. For numerical data such as blood test data and vital signs (e.g., time-series vectors for 30 days×20 items), the analysis unit applies numerical analysis algorithms such as recurrent neural networks (RNN), LSTM, and autoregressive models to output abnormal value detection and disease progression risk scores. For medical record text (e.g., “fever, cough, medical history: diabetes”), the analysis unit applies natural language processing algorithms such as large language models (LLM) and BERT-based models to output disease name labels and recommended treatment policy text. Furthermore, when categories are combined (e.g., multimodal data of images and text), the analysis unit applies multimodal AI models (e.g., Vision Transformer+LLM collaboration) to generate integrated analysis results. For training the AI models, category-optimized supervised learning (e.g., Dice loss for images, MSE loss for numerical data, cross-entropy loss for text, etc.) and optimization methods (e.g., Adam, SGD, etc.) are applied for each category. Examples of AI model output include “image→abnormal region mask, abnormality probability 0.85,” “numerical→anemia risk 0.65,” “text→disease name: pneumonia, recommended treatment: antibiotic administration.” These outputs are used for subsequent diagnostic support, automatic report generation, and electronic medical record keeping. As a technical effect, the analysis unit can automatically select and apply optimal AI algorithms for each data category, thereby improving analysis accuracy, computational efficiency, reducing misjudgments, and strengthening diagnostic support through integrated analysis of multiple data sources. Specific application fields include remote image diagnosis support, rapid triage in emergency medical settings, chronic disease management, cancer screening, and prognosis-predictive personalized medicine. Furthermore, variations of analysis algorithms such as adaptation to new categories by transfer learning, improvement of accuracy by ensemble learning, and addition of explainable AI (XAI) for evidence presentation are also possible. With these configurations, the present invention not only automates human tasks but also contributes to the improvement of computer technology itself and the resolution of issues in medical practice.

The analysis unit can estimate a patient's emotion and adjust the length of analysis based on the estimated emotion of the patient. For example, if the patient is feeling anxious, the analysis unit provides a short and concise analysis result. Emotion estimation is realized using an emotion engine or generative AI, such as emotion estimation functions. Generative AI may include text generation AI (e.g., LLM) or multimodal generative AI, but is not limited thereto. For example, the analysis unit captures the patient's facial expressions with a camera and estimates emotion using an emotion estimation algorithm. The emotion score is calculated based on changes in facial expressions, and the length of analysis is adjusted. The analysis unit can also record the patient's voice and estimate emotion using voice analysis technology. The tone and speed of the voice are analyzed, the emotion score is calculated, and the length of analysis is adjusted. Furthermore, the analysis unit can collect the patient's biometric data (heart rate and skin electrical activity) with sensors and estimate emotion using an emotion estimation algorithm. The emotion score is calculated based on heart rate variability, and the length of analysis is adjusted. Thus, by adjusting the length of analysis according to the patient's emotion, the analysis unit can provide more appropriate analysis results. Specifically, the analysis unit simultaneously acquires multiple sensor data for emotion estimation of the patient (e.g., facial image 224×224×3 RGB tensor, one-dimensional time-series array of voice waveform, numerical vector of heart rate and skin electrical activity, etc.), preprocesses these data (e.g., face region extraction from facial images, noise removal from voice, normalization of vital data), and inputs them to an AI model for emotion estimation. The analysis unit uses a convolutional neural network (CNN) for facial images, a recurrent neural network (RNN) or a model with self-attention mechanism for voice, and a multilayer perceptron (MLP) for biometric data. For example, the CNN takes a facial image tensor as input and outputs a probability distribution of emotion labels such as “joy,” “anxiety,” and “anger” (e.g., anxiety 0.75, relaxation 0.15, joy 0.10). The RNN takes a voice waveform as input and outputs an emotion score (e.g., anxiety level 0.68) from the tone and speed of the voice. The MLP takes a time-series vector of heart rate and skin electrical activity as input and outputs scores for stress level and relaxation level (e.g., stress 0.80). The analysis unit integrates the outputs of these multiple AI models and calculates the final emotion estimation result (e.g., anxiety level 0.72). The emotion estimation result is input to the analysis length control module, and the length of analysis results is automatically determined by rule-based or machine learning-based length optimization algorithms, such as “if anxiety level is 0.7 or higher, summarize analysis results within two sentences,” or “if relaxation level is 0.8 or higher, provide detailed analysis explanations.” For training the AI models, supervised learning using multimodal datasets with emotion labels (cross-entropy loss function, Adam optimization, etc.) is applied to improve estimation accuracy. Examples of AI model output include “anxiety level 0.75→output only key points in a shortened format,” “relaxation level 0.85→provide detailed evidence explanation.” These outputs are input to the control module of the analysis unit, and the actual analysis result generation process (e.g., generation of explanatory text by natural language generation AI, application of summarization algorithms, etc.) is automatically controlled. Thus, the analysis unit can integratively analyze multiple biometric and behavioral data in high-dimensional feature space without relying on subjective judgment or empirical rules of human operators, thereby minimizing psychological burden on patients and providing analysis results at the optimal length. As a technical effect, the analysis unit contributes to improved patient understanding, reduced stress during explanation, improved accuracy of medical AI analysis, and increased patient satisfaction. Specific application fields include medical explanations for children and the elderly, consideration for patients with mental disorders, stress-free explanations in remote medical settings, and reduction of subject explanation burden in clinical trials and research. Furthermore, variations of the emotion estimation AI model such as adaptation to individual characteristics by transfer learning, improvement of estimation accuracy by ensemble learning, and addition of explainable AI (XAI) for presenting the basis of emotion estimation are also possible. With these configurations, the present invention not only automates human tasks but also contributes to the improvement of computer technology itself and the resolution of issues in medical practice.

The analysis unit can determine the priority of analysis based on the submission timing of medical data during analysis. For example, the analysis unit prioritizes analysis for the latest medical data. Submission timing includes, for example, submission date and submission frequency, but is not limited thereto. For example, the analysis unit prioritizes analysis for the latest medical data. The analysis unit can also postpone analysis for older medical data. For example, the analysis unit postpones analysis for older medical data. Furthermore, the analysis unit can adjust the priority of analysis according to the submission timing of medical data. For example, the analysis unit adjusts the priority of analysis according to the submission timing of medical data. Thus, by determining the priority of analysis according to the submission timing of medical data, the analysis unit can perform analysis efficiently. Specifically, the analysis unit is equipped with a submission timing management module that automatically records and manages metadata such as submission date and submission frequency for each medical data. The analysis unit inputs submission date (e.g., timestamp), submission frequency (e.g., once a day, once a week, etc.), and data type (e.g., medical records, test results, medical images, etc.) as input vectors to a priority judgment AI model (e.g., decision tree, random forest, neural network, etc.). The AI model outputs a high priority score (e.g., 0.9) for the latest data and a low priority score (e.g., 0.2) for older data, and the analysis unit automatically controls the order of the analysis queue based on this score. For example, immediate analysis flow is applied to the latest vital data of emergency patients (submission date: current time), and postponed analysis flow is applied to past data from periodic health checkups (submission date: one month ago). For training the AI models, supervised learning using past analysis history and clinical outcomes (cross-entropy loss function, Adam optimization, etc.) is applied to improve priority judgment accuracy. Examples of AI model output include “submission date: 2024-06-01 10:00→priority 0.95,” “submission date: 2024-05-01 10:00→priority 0.30.” These outputs are input to the control module of the analysis unit, and the actual analysis process (e.g., job submission order to GPU cluster, trigger for real-time analysis, etc.) is automatically controlled. Thus, the analysis unit can realize objective and efficient control of analysis order based on submission timing, unlike manual determination of analysis order by human operators. As a technical effect, the analysis unit achieves prevention of missing urgent cases, minimization of diagnostic delay, optimal allocation of computational resources, and overall improvement of analysis efficiency. Specific application fields include real-time triage in emergency medical settings, periodic data analysis in chronic disease management, and priority analysis under bandwidth constraints in remote medical care. Furthermore, variations of the priority judgment AI model such as adaptation to new data types by transfer learning, improvement of judgment accuracy by ensemble learning, and addition of explainable AI (XAI) for presenting the basis of priority judgment are also possible. With these configurations, the present invention not only automates human tasks but also contributes to the improvement of computer technology itself and the resolution of issues in medical practice.

The analysis unit can adjust the order of analysis based on the relevance of medical data during analysis. For example, the analysis unit prioritizes analysis for highly relevant medical data. Relevance includes, for example, correlation and degree of association between data, but is not limited thereto. For example, the analysis unit prioritizes analysis for highly relevant medical data. The analysis unit can also postpone analysis for less relevant medical data. For example, the analysis unit postpones analysis for less relevant medical data. Furthermore, the analysis unit can adjust the order of analysis according to the relevance of medical data. For example, the analysis unit adjusts the order of analysis according to the relevance of medical data. Thus, by adjusting the order of analysis according to the relevance of medical data, the analysis unit can perform analysis efficiently. Specifically, the analysis unit is equipped with a relevance evaluation module that automatically calculates correlation and degree of association between multiple medical data. The analysis unit receives medical records (text data, ICD codes, etc.), test results (numerical vectors, time-series data), and medical images (three-dimensional tensors in DICOM format, etc.) as input data, and extracts relevance features between these data (e.g., same patient ID, same medical date, same disease label, correlation coefficient of test values, etc.). A relevance judgment AI model (e.g., graph neural network, decision tree, neural network, etc.) takes these features as input and outputs relevance scores (e.g., 0.0-1.0). Data groups with high relevance scores are assigned to simultaneous analysis or priority analysis flows, and data groups with low relevance scores are assigned to postponed analysis flows. For example, consecutive vital data and image data of the same patient (relevance 0.9) are analyzed simultaneously, and data of different patients or different periods (relevance 0.2) are postponed. For training the AI models, supervised learning using past diagnostic results and clinical usefulness of data relevance (cross-entropy loss function, Adam optimization, etc.) is applied to improve relevance judgment accuracy. Examples of AI model output include “relevance 0.95→simultaneous analysis, priority analysis,” “relevance 0.25→postponed analysis.” These outputs are input to the control module of the analysis unit, and the order of the analysis queue and automatic organization of simultaneous analysis groups are controlled. Thus, the analysis unit can realize automatic and objective control of analysis order based on relevance evaluation in high-dimensional feature space, unlike empirical rules and manual data grouping by human operators. As a technical effect, the analysis unit achieves improved diagnostic accuracy by simultaneous analysis of clinically important data groups, improved analysis efficiency, reduction of unnecessary duplicate analysis, and strengthened diagnostic support through integrated analysis of multiple data sources. Specific application fields include simultaneous triage of multiple items in emergency medical settings, integrated analysis of time-series data in chronic disease management, multimodality analysis in cancer screening, and priority analysis of relevant data in remote medical care. Furthermore, variations of the relevance judgment AI model such as adaptation to new diseases and data types by transfer learning, improvement of judgment accuracy by ensemble learning, and addition of explainable AI (XAI) for presenting the basis of relevance judgment are also possible. With these configurations, the present invention not only automates human tasks but also contributes to the improvement of computer technology itself and the resolution of issues in medical practice.

The provision unit is capable of estimating a patient's emotion and adjusting the method of providing a diagnostic result based on the estimated emotion of the patient. For example, when the patient is feeling anxious, the provision unit provides a simple and easy-to-understand diagnostic result. Emotion estimation is realized by using an emotion estimation function, such as an emotion engine or generative AI. Generative AI may include text generation AI (e.g., LLM) or multimodal generative AI, but is not limited thereto. For example, the provision unit may capture the patient's facial expression with a camera and estimate the emotion using an emotion estimation algorithm. Based on changes in facial expression, an emotion score is calculated and the method of providing the diagnostic result is adjusted. Additionally, the provision unit may record the patient's voice and estimate the emotion using voice analysis technology. By analyzing the tone and speed of the voice, an emotion score is calculated and the method of providing the diagnostic result is adjusted. Furthermore, the provision unit may collect the patient's biometric data (such as heart rate and skin electrical activity) with sensors and estimate the emotion using an emotion estimation algorithm. Based on heart rate variability, an emotion score is calculated and the method of providing the diagnostic result is adjusted. By adjusting the method of providing the diagnostic result according to the patient's emotion, more appropriate diagnostic results can be provided. Specifically, the provision unit simultaneously acquires multiple sensor data for emotion estimation (e.g., facial images as 224×224×3 RGB tensors, one-dimensional time-series arrays of voice waveforms, numerical vectors of heart rate and skin electrical activity, etc.), performs preprocessing (such as facial region extraction from facial images, noise removal from voice, normalization of vital data), and inputs these into an AI model for emotion estimation. For facial images, a convolutional neural network (CNN) is used; for voice, a recurrent neural network (RNN) or a model with self-attention mechanism; and for biometric data, a multilayer perceptron (MLP) is used. For example, the CNN takes the facial image tensor as input and outputs a probability distribution of emotion labels such as “joy,” “anxiety,” “anger” (e.g., anxiety 0.75, relaxation 0.15, joy 0.10). The RNN takes the voice waveform as input and outputs an emotion score (e.g., anxiety level 0.68) based on tone and speed. The MLP takes time-series vectors of heart rate and skin electrical activity as input and outputs scores for stress and relaxation (e.g., stress 0.80). The provision unit integrates the outputs of these multiple AI models to calculate a final emotion estimation result (e.g., anxiety level 0.72). The emotion estimation result is input to a diagnostic result provision control module, and, for example, rules-based or machine learning-based expression optimization algorithms automatically determine the method of providing the diagnostic result, such as “if anxiety level is 0.7 or higher, summarize the diagnostic result within two sentences and eliminate technical terms,” or “if relaxation level is 0.8 or higher, provide a detailed diagnostic explanation.” For training the AI models, supervised learning using multimodal datasets with emotion labels (cross-entropy loss function, Adam optimizer, etc.) is applied to improve estimation accuracy. Example outputs of the AI models include “anxiety level 0.75→output summarized diagnostic result,” “relaxation level 0.85→provide detailed explanation of rationale.” These outputs are input to the control module of the provision unit, which automatically controls the actual diagnostic result generation process (such as explanation text generation by natural language generation AI, automatic replacement of technical terms, etc.). Thus, the provision unit can provide diagnostic results in the optimal expression while minimizing the patient's psychological burden by integratively analyzing multiple biometric and behavioral data in a high-dimensional feature space, without relying on subjective judgment or empirical rules of human operators. The technical effects include improved patient comprehension, reduced stress during explanation, improved accuracy of medical AI analysis, and increased patient satisfaction. Specific application fields include explanations for pediatric and elderly patients, consideration for patients with mental disorders, stress-free explanations in remote medical settings, and reduction of explanation burden for subjects in clinical trials and research. Furthermore, variations of the emotion estimation AI model, such as adaptation to individual characteristics by transfer learning, improved estimation accuracy by ensemble learning, and addition of explanation capability (XAI) for presenting the basis of emotion estimation, are also possible. With these configurations, the present invention contributes not only to automation of human tasks but also to improvement of computer technology itself and to solving issues in medical practice.

The provision unit is capable of adjusting the level of detail of provision based on the importance of medical data when providing a diagnostic result. For example, the provision unit provides a detailed explanation for important diagnostic results. Importance may include, for example, the depth of information or the level of detail in the explanation, but is not limited thereto. For example, the provision unit provides a detailed explanation for important diagnostic results. Additionally, the provision unit may provide a simplified explanation for diagnostic results of low importance. For example, the provision unit provides a simplified explanation for diagnostic results of low importance. Furthermore, the provision unit may adjust the level of detail of provision according to the importance of medical data. For example, the provision unit adjusts the level of detail of provision according to the importance of medical data. By adjusting the level of detail of provision according to the importance of medical data, the provision unit can efficiently provide diagnostic results. Specifically, the provision unit is equipped with an importance evaluation module that automatically calculates an importance score for each diagnostic result, such as urgency or clinical impact. The provision unit receives input data such as medical records (text data, ICD codes, etc.), test results (numerical vectors, time-series data), and medical images (three-dimensional tensors in DICOM format, etc.), and applies an importance determination AI model (e.g., decision tree, random forest, neural network, etc.) to these data. The AI model extracts features such as “vital abnormality in emergency transport patient,” “image suspected of tumor,” “rapid change in test values,” and outputs an importance score (e.g., 0.0 to 1.0). When the importance score is high, the provision unit automatically generates a detailed explanation of the diagnostic result (e.g., rationale for abnormal region, reason for treatment policy selection, comparison with past cases, etc.) and provides it to the physician or patient. When the importance is low, a concise summary of the key points is generated. Example outputs of the AI model include “importance 0.95→provide detailed explanation,” “importance 0.25→provide summary explanation only.” These outputs are input to the control module of the provision unit, which automatically controls the explanation generation process for diagnostic results (such as detailed explanation generation by natural language generation AI, application of summarization algorithms, etc.). For training the AI model, supervised learning using past diagnostic results and clinical outcomes as training data (cross-entropy loss function, Adam optimizer, etc.) is applied to improve the accuracy of importance determination. As a result, the provision unit achieves improved speed of explanation generation by optimal allocation of computational resources, prevention of missing important cases, reduction of unnecessary detailed explanations and associated costs, and overall improvement in the accuracy of diagnostic explanations. Specific application fields include triage support in emergency medical settings, early detection of abnormalities in chronic disease management, prioritized explanation in cancer screening, and efficient explanation under bandwidth constraints in remote medical care. Furthermore, variations of the importance determination AI model, such as adaptation to new diseases by transfer learning, improved determination accuracy by ensemble learning, and addition of explanation capability (XAI) for presenting the basis of importance, are also possible. With these configurations, the present invention contributes not only to automation of human tasks but also to improvement of computer technology itself and to solving issues in medical practice.

The provision unit is capable of applying different provision algorithms according to the category of medical data when providing a diagnostic result. For example, the provision unit provides image analysis results for medical image data. Categories may include, for example, data classification methods or provision means, but are not limited thereto. For example, the provision unit provides image analysis results for medical image data. Additionally, the provision unit may provide numerical analysis results for blood test data. For example, the provision unit provides numerical analysis results for blood test data. Furthermore, the provision unit may provide text analysis results for medical record data. For example, the provision unit provides text analysis results for medical record data. By applying appropriate provision algorithms according to the category of medical data, the provision unit can improve the accuracy of diagnostic results. Specifically, the provision unit is equipped with a data classification module that automatically determines the category of input data (e.g., medical images, test values, medical record text, vital time-series data, etc.). For medical images (e.g., DICOM format X-ray images 224×224×3, MRI images 256×256×128, etc.), the provision unit provides abnormal region masks and abnormal probability scores using image analysis AI models (CNN, U-Net, etc.). For numerical data such as blood test data and vital signs (e.g., time-series vectors of 30 days×20 items), the provision unit provides abnormal value detection and disease progression risk scores using numerical analysis AI models (RNN, LSTM, etc.). For medical record text (e.g., “fever, cough, medical history: diabetes”), the provision unit provides disease name labels and recommended treatment policy text using natural language processing AI models (LLM, BERT-based models, etc.). Furthermore, when categories are combined (e.g., multimodal data of image+text), the provision unit applies multimodal AI models (e.g., Vision Transformer+LLM collaboration) to generate and provide integrated diagnostic results. For training the AI models, category-optimized supervised learning (e.g., Dice loss for images, MSE loss for numerical data, cross-entropy loss for text, etc.) and optimization methods (e.g., Adam, SGD, etc.) are applied for each category. Example outputs of the AI models include “image→abnormal region mask, abnormal probability 0.85,” “numerical→anemia risk 0.65,” “text→disease name: pneumonia, recommended treatment: antibiotic administration.” These outputs are used for subsequent diagnostic support, automatic report generation, electronic medical record documentation, etc. The technical effects include improved accuracy of diagnostic result provision, computational efficiency, reduction of misdiagnosis, and enhanced diagnostic support through integrated analysis of multiple data sources by automatically selecting and applying optimal AI algorithms for each data category. Specific application fields include remote image diagnosis support, rapid triage in emergency medical settings, chronic disease management, cancer screening, and prognosis-predictive personalized medicine. Furthermore, variations of the provision algorithms, such as adaptation to new categories by transfer learning, improved accuracy by ensemble learning, and addition of explanation capability (XAI) for presenting the basis of provision, are also possible. With these configurations, the present invention contributes not only to automation of human tasks but also to improvement of computer technology itself and to solving issues in medical practice.

The provision unit is capable of estimating a patient's emotion and adjusting the length of a diagnostic result based on the estimated emotion of the patient when providing a diagnostic result. For example, when the patient is feeling anxious, the provision unit provides a short diagnostic result that covers the key points. Emotion estimation is realized by using an emotion estimation function, such as an emotion engine or generative AI. Generative AI may include text generation AI (e.g., LLM) or multimodal generative AI, but is not limited thereto. For example, the provision unit may capture the patient's facial expression with a camera and estimate the emotion using an emotion estimation algorithm. Based on changes in facial expression, an emotion score is calculated and the length of the diagnostic result is adjusted. Additionally, the provision unit may record the patient's voice and estimate the emotion using voice analysis technology. By analyzing the tone and speed of the voice, an emotion score is calculated and the length of the diagnostic result is adjusted. Furthermore, the provision unit may collect the patient's biometric data (such as heart rate and skin electrical activity) with sensors and estimate the emotion using an emotion estimation algorithm. Based on heart rate variability, an emotion score is calculated and the length of the diagnostic result is adjusted. By adjusting the length of the diagnostic result according to the patient's emotion, more appropriate diagnostic results can be provided. Specifically, the provision unit simultaneously acquires multiple sensor data for emotion estimation (e.g., facial images as 224×224×3 RGB tensors, one-dimensional time-series arrays of voice waveforms, numerical vectors of heart rate and skin electrical activity, etc.), performs preprocessing (such as facial region extraction from facial images, noise removal from voice, normalization of vital data), and inputs these into an AI model for emotion estimation. For facial images, a convolutional neural network (CNN) is used; for voice, a recurrent neural network (RNN) or a model with self-attention mechanism; and for biometric data, a multilayer perceptron (MLP) is used. For example, the CNN takes the facial image tensor as input and outputs a probability distribution of emotion labels such as “joy,” “anxiety,” “anger” (e.g., anxiety 0.75, relaxation 0.15, joy 0.10). The RNN takes the voice waveform as input and outputs an emotion score (e.g., anxiety level 0.68) based on tone and speed. The MLP takes time-series vectors of heart rate and skin electrical activity as input and outputs scores for stress and relaxation (e.g., stress 0.80). The provision unit integrates the outputs of these multiple AI models to calculate a final emotion estimation result (e.g., anxiety level 0.72). The emotion estimation result is input to a diagnostic result length control module, and, for example, rules-based or machine learning-based length optimization algorithms automatically determine the length of the diagnostic result, such as “if anxiety level is 0.7 or higher, summarize the diagnostic result within two sentences,” or “if relaxation level is 0.8 or higher, provide a detailed diagnostic explanation.” For training the AI models, supervised learning using multimodal datasets with emotion labels (cross-entropy loss function, Adam optimizer, etc.) is applied to improve estimation accuracy. Example outputs of the AI models include “anxiety level 0.75→output shortened diagnostic result with key points only,” “relaxation level 0.85→provide detailed explanation of rationale.” These outputs are input to the control module of the provision unit, which automatically controls the actual diagnostic result generation process (such as explanation text generation by natural language generation AI, application of summarization algorithms, etc.). Thus, the provision unit can provide diagnostic results in the optimal length while minimizing the patient's psychological burden by integratively analyzing multiple biometric and behavioral data in a high-dimensional feature space, without relying on subjective judgment or empirical rules of human operators. The technical effects include improved patient comprehension, reduced stress during explanation, improved accuracy of medical AI analysis, and increased patient satisfaction. Specific application fields include explanations for pediatric and elderly patients, consideration for patients with mental disorders, stress-free explanations in remote medical settings, and reduction of explanation burden for subjects in clinical trials and research. Furthermore, variations of the emotion estimation AI model, such as adaptation to individual characteristics by transfer learning, improved estimation accuracy by ensemble learning, and addition of explanation capability (XAI) for presenting the basis of emotion estimation, are also possible. With these configurations, the present invention contributes not only to automation of human tasks but also to improvement of computer technology itself and to solving issues in medical practice.

The provision unit is capable of determining the priority of provision based on the submission timing of medical data when providing a diagnostic result. For example, the provision unit gives priority to the latest diagnostic results. Submission timing may include, for example, submission date or submission frequency, but is not limited thereto. For example, the provision unit gives priority to the latest diagnostic results. Additionally, the provision unit may postpone the provision of older diagnostic results. For example, the provision unit postpones the provision of older diagnostic results. Furthermore, the provision unit may adjust the priority of provision according to the submission timing of medical data. For example, the provision unit adjusts the priority of provision according to the submission timing of medical data. By determining the priority of provision according to the submission timing of medical data, the provision unit can efficiently provide diagnostic results. Specifically, the provision unit is equipped with a submission timing management module that automatically records and manages metadata such as submission date and submission frequency for each diagnostic result. The provision unit inputs submission date (e.g., timestamp), submission frequency (e.g., once per day, once per week, etc.), and data type (e.g., medical records, test results, medical images, etc.) as input vectors to a priority determination AI model (e.g., decision tree, random forest, neural network, etc.). The AI model outputs a high priority score (e.g., 0.9) for the latest data and a low priority score (e.g., 0.2) for older data, and the provision unit automatically controls the order of the diagnostic result provision queue based on this score. For example, immediate provision flow is applied to the most recent vital data of an emergency patient (submission date: current time), and postponed provision flow is applied to past data from a regular checkup (submission date: one month ago). For training the AI model, supervised learning using past diagnostic result provision history and clinical outcomes as training data (cross-entropy loss function, Adam optimizer, etc.) is applied to improve the accuracy of priority determination. Example outputs of the AI model include “submission date: 2024-06-01 10:00→priority 0.95,” “submission date: 2024-05-01 10:00→priority 0.30.” These outputs are input to the control module of the provision unit, which automatically controls the actual diagnostic result provision process (e.g., control of notification timing, prioritization of output destination, etc.). Thus, the provision unit can realize objective and efficient control of the order of diagnostic result provision based on submission timing, unlike manual determination of provision order by human operators. The technical effects include prevention of missing urgent cases, minimization of diagnostic delays, optimal allocation of computational resources, and overall improvement in the efficiency of diagnostic result provision. Specific application fields include real-time triage in emergency medical settings, regular data provision in chronic disease management, and prioritized provision under bandwidth constraints in remote medical care. Furthermore, variations of the priority determination AI model, such as adaptation to new data types by transfer learning, improved determination accuracy by ensemble learning, and addition of explanation capability (XAI) for presenting the basis of priority, are also possible. With these configurations, the present invention contributes not only to automation of human tasks but also to improvement of computer technology itself and to solving issues in medical practice.

The provision unit is capable of adjusting the order of provision based on the relevance of medical data when providing a diagnostic result. For example, the provision unit gives priority to diagnostic results with high relevance. Relevance may include, for example, data correlation or degree of relevance, but is not limited thereto. For example, the provision unit gives priority to diagnostic results with high relevance. Additionally, the provision unit may postpone the provision of diagnostic results with low relevance. For example, the provision unit postpones the provision of diagnostic results with low relevance. Furthermore, the provision unit may adjust the order of provision according to the relevance of medical data. For example, the provision unit adjusts the order of provision according to the relevance of medical data. By adjusting the order of provision according to the relevance of medical data, the provision unit can efficiently provide diagnostic results. Specifically, the provision unit is equipped with a relevance evaluation module that automatically calculates the correlation or degree of relevance between multiple diagnostic results. The provision unit receives input data such as medical records (text data, ICD codes, etc.), test results (numerical vectors, time-series data), and medical images (three-dimensional tensors in DICOM format, etc.), and extracts relevance features between these data (e.g., same patient ID, same medical date, same disease label, correlation coefficient of test values, etc.). A relevance determination AI model (e.g., graph neural network, decision tree, neural network, etc.) takes these features as input and outputs a relevance score (e.g., 0.0 to 1.0). Diagnostic result groups with high relevance scores are assigned to simultaneous provision or priority provision flows, while groups with low relevance scores are assigned to postponed provision flows. For example, consecutive vital data and image diagnostic results for the same patient (relevance 0.9) are provided simultaneously, while diagnostic results for different patients or different periods (relevance 0.2) are postponed. For training the AI model, supervised learning using past diagnostic results and the clinical usefulness of data relevance as training data (cross-entropy loss function, Adam optimizer, etc.) is applied to improve the accuracy of relevance determination. Example outputs of the AI model include “relevance 0.95→simultaneous provision, priority provision,” “relevance 0.25→postponed provision.” These outputs are input to the control module of the provision unit, which controls the order of the diagnostic result provision queue and the automatic organization of simultaneous provision groups. Thus, the provision unit can realize automatic and objective control of provision order based on relevance evaluation in a high-dimensional feature space, unlike grouping of diagnostic results by human operators based on experience or manual work. The technical effects include improved diagnostic accuracy by simultaneous provision of clinically important diagnostic result groups, improved provision efficiency, reduction of unnecessary duplicate explanations, and enhanced diagnostic support through integrated explanation of multiple data sources. Specific application fields include simultaneous multi-item triage in emergency medical settings, integrated explanation of time-series data in chronic disease management, multimodality explanation in cancer screening, and prioritized provision of relevant data in remote medical care. Furthermore, variations of the relevance determination AI model, such as adaptation to new diseases or data types by transfer learning, improved determination accuracy by ensemble learning, and addition of explanation capability (XAI) for presenting the basis of relevance, are also possible. With these configurations, the present invention contributes not only to automation of human tasks but also to improvement of computer technology itself and to solving issues in medical practice.

The remote support unit is capable of estimating a patient's emotion and adjusting the method of remote support based on the estimated emotion of the patient. For example, when the patient is feeling anxious, the remote support unit provides a support method that helps the patient relax. Emotion estimation is realized by using an emotion estimation function, such as an emotion engine or generative AI. Generative AI may include text generation AI (e.g., LLM) or multimodal generative AI, but is not limited thereto. For example, the remote support unit may capture the patient's facial expression with a camera and estimate the emotion using an emotion estimation algorithm. Based on changes in facial expression, an emotion score is calculated and the method of remote support is adjusted. Additionally, the remote support unit may record the patient's voice and estimate the emotion using voice analysis technology. By analyzing the tone and speed of the voice, an emotion score is calculated and the method of remote support is adjusted. Furthermore, the remote support unit may collect the patient's biometric data (such as heart rate and skin electrical activity) with sensors and estimate the emotion using an emotion estimation algorithm. Based on heart rate variability, an emotion score is calculated and the method of remote support is adjusted. By adjusting the method of remote support according to the patient's emotion, more appropriate support can be provided.

The remote support unit is capable of estimating a patient's emotion and determining the priority of remote support based on the estimated emotion of the patient. For example, when the patient is feeling anxious, the remote support unit provides support with priority. Emotion estimation is realized by using an emotion estimation function, such as an emotion engine or generative AI. Generative AI may include text generation AI (e.g., LLM) or multimodal generative AI, but is not limited thereto. For example, the remote support unit may capture the patient's facial expression with a camera and estimate the emotion using an emotion estimation algorithm. Based on changes in facial expression, an emotion score is calculated and the priority of remote support is determined. Additionally, the remote support unit may record the patient's voice and estimate the emotion using voice analysis technology. By analyzing the tone and speed of the voice, an emotion score is calculated and the priority of remote support is determined. Furthermore, the remote support unit may collect the patient's biometric data (such as heart rate and skin electrical activity) with sensors and estimate the emotion using an emotion estimation algorithm. Based on heart rate variability, an emotion score is calculated and the priority of remote support is determined. By determining the priority of remote support according to the patient's emotion, more appropriate support can be provided.

The remote support unit is capable of selecting an optimal support method by considering the patient's geographic location information during remote support. For example, the remote support unit selects an optimal support method based on the patient's geographic location information. Geographic location information may include, for example, GPS data or location information services, but is not limited thereto. For example, the remote support unit adjusts the support content based on the patient's geographic location information. Geographic location information may include, for example, the patient's residence or medical treatment location. For example, the remote support unit adjusts the support content based on the patient's geographic location information. Additionally, the remote support unit may determine the priority of support based on the patient's geographic location information. For example, the remote support unit determines the priority of support based on the patient's geographic location information. By considering the patient's geographic location information, the remote support unit can select an optimal support method.

The rapid diagnosis unit is capable of estimating a patient's emotion and adjusting the method of rapid diagnosis based on the estimated emotion of the patient. For example, when the patient is feeling anxious, the rapid diagnosis unit provides a simple and easy-to-understand rapid diagnosis method. Emotion estimation is realized by using an emotion estimation function, such as an emotion engine or generative AI. Generative AI may include text generation AI (e.g., LLM) or multimodal generative AI, but is not limited thereto. For example, the rapid diagnosis unit may capture the patient's facial expression with a camera and estimate the emotion using an emotion estimation algorithm. Based on changes in facial expression, an emotion score is calculated and the method of rapid diagnosis is adjusted. Additionally, the rapid diagnosis unit may record the patient's voice and estimate the emotion using voice analysis technology. By analyzing the tone and speed of the voice, an emotion score is calculated and the method of rapid diagnosis is adjusted. Furthermore, the rapid diagnosis unit may collect the patient's biometric data (such as heart rate and skin electrical activity) with sensors and estimate the emotion using an emotion estimation algorithm. Based on heart rate variability, an emotion score is calculated and the method of rapid diagnosis is adjusted. By adjusting the method of rapid diagnosis according to the patient's emotion, more appropriate diagnosis can be provided.

The rapid diagnosis unit is capable of selecting an optimal diagnostic method by referring to the patient's past medical records during rapid diagnosis. For example, the rapid diagnosis unit selects an optimal rapid diagnostic method based on the patient's past medical records. Diagnostic methods may include, for example, diagnostic means or diagnostic algorithms, but are not limited thereto. For example, the rapid diagnosis unit adjusts the diagnostic content based on the patient's past medical records. Past medical records may include diagnostic results or treatment history. For example, the rapid diagnosis unit adjusts the diagnostic content based on the patient's past medical records. Additionally, the rapid diagnosis unit may analyze the patient's past medical records and determine the priority of diagnosis. For example, the rapid diagnosis unit analyzes the patient's past medical records and determines the priority of diagnosis. By referring to the patient's past medical records, the rapid diagnosis unit can select an optimal rapid diagnostic method.

The rapid diagnosis unit is capable of estimating a patient's emotion and determining the priority of rapid diagnosis based on the estimated emotion of the patient. For example, when the patient is feeling anxious, the rapid diagnosis unit provides rapid diagnosis with priority. Emotion estimation is realized by using an emotion estimation function, such as an emotion engine or generative AI. Generative AI may include text generation AI (e.g., LLM) or multimodal generative AI, but is not limited thereto. For example, the rapid diagnosis unit may capture the patient's facial expression with a camera and estimate the emotion using an emotion estimation algorithm. Based on changes in facial expression, an emotion score is calculated and the priority of rapid diagnosis is determined. Additionally, the rapid diagnosis unit may record the patient's voice and estimate the emotion using voice analysis technology. By analyzing the tone and speed of the voice, an emotion score is calculated and the priority of rapid diagnosis is determined. Furthermore, the rapid diagnosis unit may collect the patient's biometric data (such as heart rate and skin electrical activity) with sensors and estimate the emotion using an emotion estimation algorithm. Based on heart rate variability, an emotion score is calculated and the priority of rapid diagnosis is determined. By determining the priority of rapid diagnosis according to the patient's emotion, more appropriate diagnosis can be provided.

The rapid diagnosis unit is capable of selecting an optimal diagnostic method by considering the patient's geographic location information during rapid diagnosis. For example, the rapid diagnosis unit selects an optimal rapid diagnostic method based on the patient's geographic location information. Geographic location information may include, for example, GPS data or location information services, but is not limited thereto. For example, the rapid diagnosis unit adjusts the diagnostic content based on the patient's geographic location information. Geographic location information may include, for example, the patient's residence or medical treatment location. For example, the rapid diagnosis unit adjusts the diagnostic content based on the patient's geographic location information. Additionally, the rapid diagnosis unit may determine the priority of diagnosis based on the patient's geographic location information. For example, the rapid diagnosis unit determines the priority of diagnosis based on the patient's geographic location information. By considering the patient's geographic location information, the rapid diagnosis unit can select an optimal rapid diagnostic method.

The system according to the embodiment is not limited to the examples described above, and various modifications are possible, for example, as follows.

The medical information management system is capable of collecting a patient's lifestyle habit data and transmitting it to the analysis unit. For example, the collection unit acquires records of the patient's diet and exercise and transmits these data to the analysis unit. The analysis unit can analyze the patient's health condition in more detail based on these data. Additionally, the collection unit may monitor the patient's sleep patterns and stress levels and transmit these data to the analysis unit. Thus, the medical information management system can realize more comprehensive health management by utilizing the patient's lifestyle habit data.

The medical information management system is capable of estimating a patient's emotion and adjusting the method of analyzing medical data based on the estimated emotion. For example, when the patient is feeling anxious, the analysis unit provides a simple and easy-to-understand analysis result. Additionally, when the patient is relaxed, the analysis unit may provide a detailed analysis result. Furthermore, the analysis unit may adjust the method of presenting the analysis result according to the patient's emotion. Thus, the medical information management system can provide analysis results that take the patient's emotion into consideration.

The medical information management system is capable of recommending an optimal medical institution by considering the patient's geographic location information. For example, the provision unit recommends the nearest medical institution based on the patient's residence or medical treatment location. Additionally, the provision unit may recommend a medical institution with a short waiting time for medical treatment based on the patient's geographic location information. Furthermore, the provision unit may recommend a medical institution capable of responding in emergencies according to the patient's geographic location information. Thus, the medical information management system can recommend an optimal medical institution by utilizing the patient's geographic location information.

The medical information management system is capable of analyzing a patient's social media activity and collecting relevant medical data. For example, the collection unit analyzes the patient's social media posts and collects health-related information. Additionally, the type of data to be collected may be adjusted based on the patient's social media activity. Furthermore, the collection unit may determine the priority of data to be collected based on the patient's social media activity. Thus, the medical information management system can efficiently collect relevant medical data by utilizing the patient's social media activity.

The medical information management system is capable of estimating a patient's emotion and adjusting the method of providing a diagnostic result based on the estimated emotion. For example, when the patient is feeling anxious, the provision unit provides a simple and easy-to-understand diagnostic result. Additionally, when the patient is relaxed, the provision unit may provide a detailed diagnostic result. Furthermore, the provision unit may adjust the method of presenting the diagnostic result according to the patient's emotion. Thus, the medical information management system can provide diagnostic results that take the patient's emotion into consideration.

The medical information management system is capable of analyzing a patient's past medical records and proposing an optimal treatment method. For example, the analysis unit proposes an optimal treatment method based on the patient's past medical records. Additionally, the priority of treatment may be determined based on the patient's past medical records. Furthermore, the analysis unit may analyze the patient's past medical records and predict the effectiveness of treatment. Thus, the medical information management system can propose an optimal treatment method by utilizing the patient's past medical records.

The medical information management system can estimate a patient's emotion and adjust the method of remote support based on the estimated emotion. For example, the remote support unit provides a relaxing support method when the patient feels anxious. In addition, when the patient is relaxed, a more detailed support method can be provided. Furthermore, the remote support unit can adjust the support content according to the patient's emotion. Thus, the medical information management system can provide remote support that takes the patient's emotion into consideration.

The medical information management system can select an optimal diagnostic method by considering the patient's geographic location information. For example, the rapid diagnosis unit selects the optimal diagnostic method based on the patient's residence or the location of medical examination. The priority of diagnosis can also be determined based on the patient's geographic location information. Furthermore, the rapid diagnosis unit can adjust the diagnostic content according to the patient's geographic location information. Thus, the medical information management system can utilize the patient's geographic location information to select the optimal diagnostic method.

The medical information management system can estimate a patient's emotion and determine the priority of rapid diagnosis based on the estimated emotion. For example, the rapid diagnosis unit provides rapid diagnosis with priority when the patient feels anxious. In addition, when the patient is relaxed, a normal diagnosis can be provided. Furthermore, the rapid diagnosis unit can adjust the priority of diagnosis according to the patient's emotion. Thus, the medical information management system can provide rapid diagnosis that takes the patient's emotion into consideration.

The medical information management system can collect a patient's lifestyle habit data and transmit it to the analysis unit. For example, the collection unit acquires records of the patient's diet and exercise and transmits these data to the analysis unit. The analysis unit can analyze the patient's health condition in more detail based on these data. In addition, the collection unit can monitor the patient's sleep patterns and stress levels and transmit these data to the analysis unit. Thus, the medical information management system can utilize the patient's lifestyle habit data to achieve more comprehensive health management.

The following is a brief description of the processing flow of Example of the Embodiment.

Step 1: The collection unit collects personal medical information. Personal medical information includes medical records, test results, and medical images. The collection unit acquires the patient's medical records from an electronic medical record system and stores them in a database. Test results are automatically acquired from a testing institution and stored in the database. Medical images are acquired from an image management system of a medical institution and stored in the database.

Step 2: The analysis unit analyzes the medical data collected by the collection unit using AI. The analysis includes detection of abnormalities and support for diagnosis. For example, the analysis unit analyzes X-ray images using AI to detect abnormalities in the lungs. MRI images are analyzed using AI to detect abnormalities in the brain. Blood test results are analyzed using AI to detect abnormal values.

Step 3: The provision unit provides a diagnostic result obtained by the analysis unit. Provision includes notification to physicians and feedback to patients. For example, the provision unit notifies the physician of the diagnostic result obtained by the analysis unit for reference in diagnosis. The diagnostic result is fed back to the patient and used to determine the treatment policy. The diagnostic result is recorded in the electronic medical record system and utilized for future medical care.

The specific processing unit 290 sends the results of specific processing to the smart device 14. In the smart device 14, the control unit 46A causes the output device 40 to output the results of specific processing. The microphone 38B acquires voice indicating user input in response to the results of specific processing. The control unit 46A sends the voice data indicating user input acquired by the microphone 38B to the data processing device 12. In the data processing device 12, the specific processing unit 290 acquires the voice data.

The data generation model 58 is a so-called generative AI (Artificial Intelligence). An example of the data generation model 58 is a generative AI such as ChatGPT (registered trademark) (Internet search <URL: https://openai.com/blog/chatgpt>). The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

Moreover, the processing by the data processing system 10 described above is executed by the specific processing unit 290 of the data processing device 12 or the control unit 46A of the smart device 14, but it may be executed by both the specific processing unit 290 of the data processing device 12 and the control unit 46A of the smart device 14. Additionally, the specific processing unit 290 of the data processing device 12 acquires or collects necessary information for processing from the smart device 14 or external devices, and the smart device 14 acquires or collects necessary information for processing from the data processing device 12 or external devices.

Each of the above-mentioned elements, including the collection unit, analysis unit, provision unit, remote support unit, and rapid diagnosis unit, is implemented by at least one of, for example, the smart device 14 and the data processing apparatus 12. For example, the collection unit is implemented by a control unit 46A of the smart device 14 and acquires a patient's medical records and test results from an electronic medical record system. The analysis unit is implemented, for example, by a specific processing unit 290 of the data processing apparatus 12 and analyzes medical data using AI. The provision unit is implemented, for example, by the control unit 46A of the smart device 14 and notifies the diagnostic result to a physician or patient. The remote support unit is implemented, for example, by the specific processing unit 290 of the data processing apparatus 12 and collects medical data from a remote medical institution and provides the analysis result. The rapid diagnosis unit is implemented, for example, by the control unit 46A of the smart device 14 and promptly provides a diagnostic result to a patient who requires time to wait for diagnosis. The correspondence between each unit and the device or control unit is not limited to the above examples and various modifications are possible.

### Second Embodiment

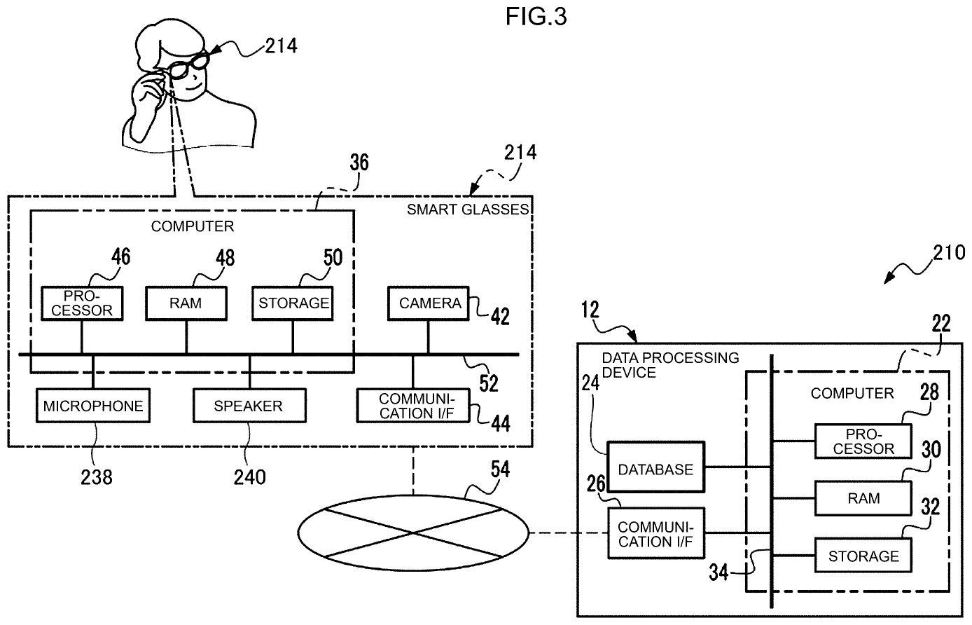

FIG. 3 shows an example configuration of a data processing system 210 according to the second embodiment.

As shown in FIG. 3, the data processing system 210 comprises a data processing device 12 and smart glasses 214. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The smart glasses 214 comprise a computer 36, a microphone 238, a speaker 240, a camera 42, and a communication I/F 44. The computer 36 comprises a processor 46, RAM 48, and storage 50. The processor 46, RAM 48, and storage 50 are connected to a bus 52. The microphone 238, speaker 240, and camera 42 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

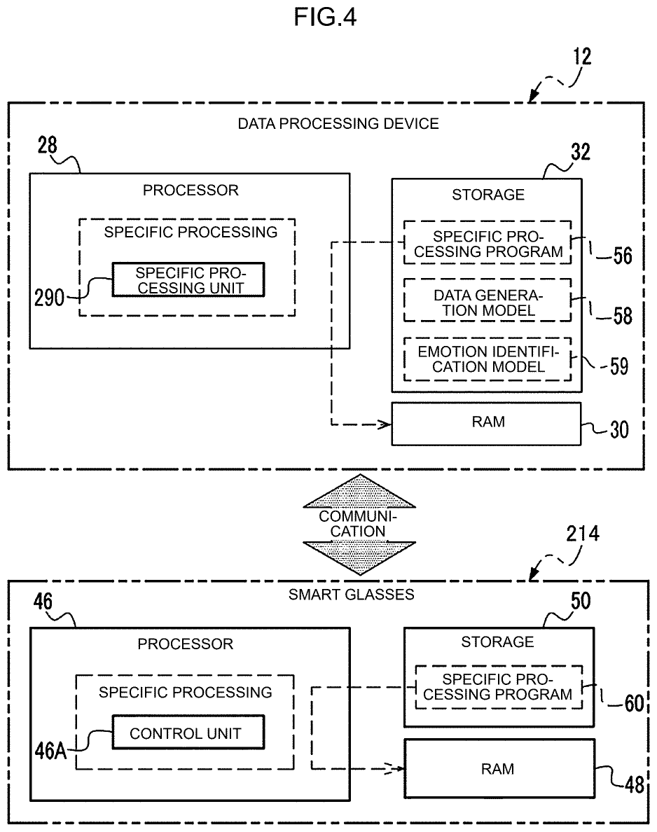

FIG. 4 shows an example of the main functions of the data processing device 12 and smart glasses 214. As shown in FIG. 4, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart glasses 214, specific processing is performed by the processor 46. The storage 50 stores a specific processing program 60. The processor 46 reads the specific processing program 60 from the storage 50 and executes it on the RAM 48. The specific processing is realized by the processor 46 operating as a control unit 46A according to the specific processing program 60 executed on the RAM 48. The smart glasses 214 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the smart glasses 214. In the smart glasses 214, the control unit 46A causes the speaker 240 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 210 according to the second embodiment performs the same processing as the data processing system 10 according to the first embodiment. The processing by the data processing system 210 is executed by the specific processing unit 290 of the data processing device 12 or the control unit 46A of the smart glasses 214, but it may be executed by both the specific processing unit 290 of the data processing device 12 and the control unit 46A of the smart glasses 214. Additionally, the specific processing unit 290 of the data processing device 12 acquires or collects necessary information for processing from the smart glasses 214 or external devices, and the smart glasses 214 acquires or collects necessary information for processing from the data processing device 12 or external devices.

Each of the above-mentioned elements, including the collection unit, analysis unit, provision unit, remote support unit, and rapid diagnosis unit, is implemented by at least one of, for example, the smart glasses 214 and the data processing apparatus 12. For example, the collection unit is implemented by a control unit 46A of the smart glasses 214 and acquires a patient's medical records and test results from an electronic medical record system. The analysis unit is implemented, for example, by a specific processing unit 290 of the data processing apparatus 12 and analyzes medical data using AI. The provision unit is implemented, for example, by the control unit 46A of the smart glasses 214 and notifies the diagnostic result to a physician or patient. The remote support unit is implemented, for example, by the specific processing unit 290 of the data processing apparatus 12 and collects medical data from a remote medical institution and provides the analysis result. The rapid diagnosis unit is implemented, for example, by the control unit 46A of the smart glasses 214 and promptly provides a diagnostic result to a patient who requires time to wait for diagnosis. The correspondence between each unit and the device or control unit is not limited to the above examples and various modifications are possible.

### Third Embodiment

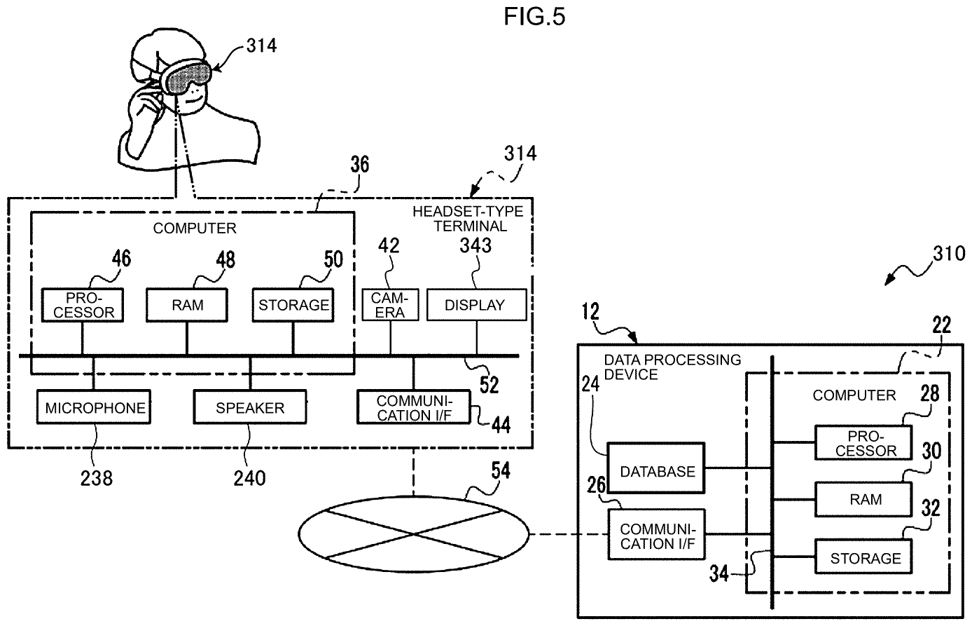

FIG. 5 shows an example configuration of a data processing system 310 according to the third embodiment.

As shown in FIG. 5, the data processing system 310 comprises a data processing device 12 and a headset-type terminal 314. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The headset-type terminal 314 comprises a computer 36, a microphone 238, a speaker 240, a camera 42, a communication I/F 44, and a display 343. The computer 36 comprises a processor 46, RAM 48, and storage 50. The processor 46, RAM 48, and storage 50 are connected to a bus 52. The microphone 238, speaker 240, camera 42, and display 343 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

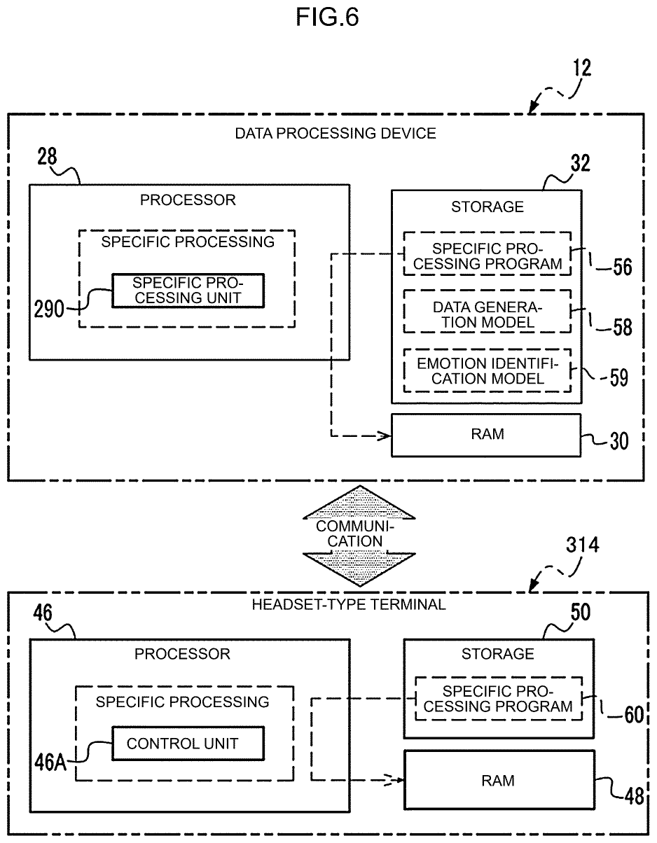

FIG. 6 shows an example of the main functions of the data processing device 12 and the headset-type terminal 314. As shown in FIG. 6, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the headset-type terminal 314, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The headset-type terminal 314 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the headset-type terminal 314. In the headset-type terminal 314, the control unit 46A causes the speaker 240 and the display 343 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 310 according to the third embodiment performs the same processing as the data processing system 10 according to the first embodiment. The processing by the data processing system 310 is executed by the specific processing unit 290 of the data processing device 12 or the control unit 46A of the headset-type terminal 314, but it may be executed by both the specific processing unit 290 of the data processing device 12 and the control unit 46A of the headset-type terminal 314. Additionally, the specific processing unit 290 of the data processing device 12 acquires or collects necessary information for processing from the headset-type terminal 314 or external devices, and the headset-type terminal 314 acquires or collects necessary information for processing from the data processing device 12 or external devices.

Each of the above-mentioned elements, including the collection unit, analysis unit, provision unit, remote support unit, and rapid diagnosis unit, is implemented by at least one of, for example, the headset-type terminal 314 and the data processing apparatus 12. For example, the collection unit is implemented by a control unit 46A of the headset-type terminal 314 and acquires a patient's medical records and test results from an electronic medical record system. The analysis unit is implemented, for example, by a specific processing unit 290 of the data processing apparatus 12 and analyzes medical data using AI. The provision unit is implemented, for example, by the control unit 46A of the headset-type terminal 314 and notifies the diagnostic result to a physician or patient. The remote support unit is implemented, for example, by the specific processing unit 290 of the data processing apparatus 12 and collects medical data from a remote medical institution and provides the analysis result. The rapid diagnosis unit is implemented, for example, by the control unit 46A of the headset-type terminal 314 and promptly provides a diagnostic result to a patient who requires time to wait for diagnosis. The correspondence between each unit and the device or control unit is not limited to the above examples and various modifications are possible.

### Fourth Embodiment

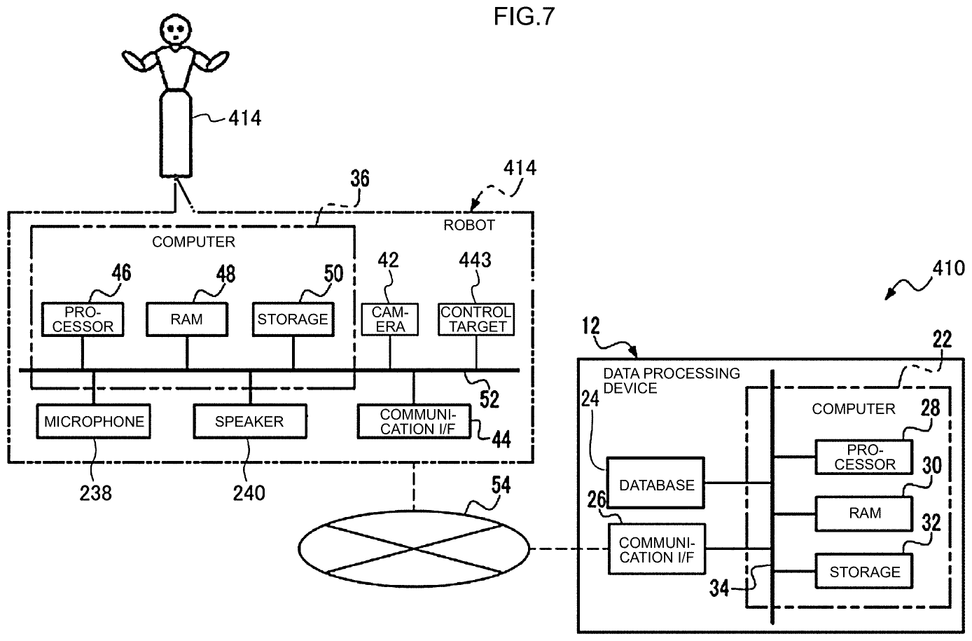

FIG. 7 shows an example configuration of a data processing system 410 according to the fourth embodiment.

As shown in FIG. 7, the data processing system 410 comprises a data processing device 12 and a robot 414. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The robot 414 comprises a computer 36, a microphone 238, a speaker 240, a camera 42, a communication I/F 44, and a control target 443. The computer 36 comprises a processor 46, RAM 48, and storage 50. The processor 46, RAM 48, and storage 50 are connected to a bus 52. The microphone 238, speaker 240, camera 42, and control target 443 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS image sensors or CCD image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

The control target 443 includes a display device, LEDs for the eyes, and motors for driving arms, hands, and feet, among others. The posture and gestures of the robot 414 are controlled by controlling the motors for the arms, hands, and feet, among others. Some emotions of the robot 414 can be expressed by controlling these motors. Additionally, the expression of the robot 414 can be expressed by controlling the lighting state of the LEDs for the eyes of the robot 414.

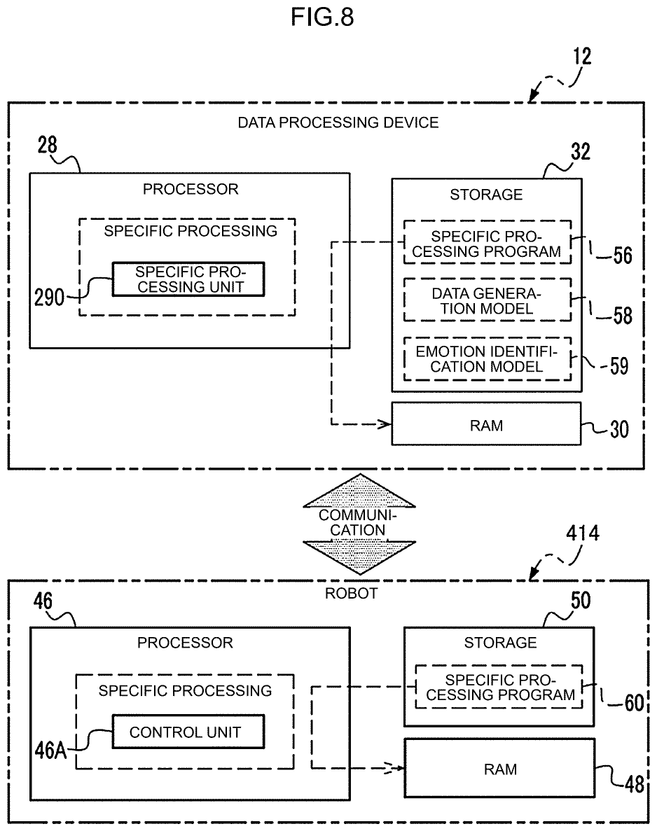

FIG. 8 shows an example of the main functions of the data processing device 12 and the robot 414. As shown in FIG. 8, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the robot 414, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The robot 414 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the robot 414. In the robot 414, the control unit 46A causes the speaker 240 and the control target 443 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 410 according to the fourth embodiment performs the same processing as the data processing system 10 according to the first embodiment. The processing by the data processing system 410 is executed by the specific processing unit 290 of the data processing device 12 or the control unit 46A of the robot 414, but it may be executed by both the specific processing unit 290 of the data processing device 12 and the control unit 46A of the robot 414. Additionally, the specific processing unit 290 of the data processing device 12 acquires or collects necessary information for processing from the robot 414 or external devices, and the robot 414 acquires or collects necessary information for processing from the data processing device 12 or external devices.

Each of the above-mentioned elements, including the collection unit, analysis unit, provision unit, remote support unit, and rapid diagnosis unit, is implemented by at least one of, for example, the robot 414 and the data processing apparatus 12. For example, the collection unit is implemented by a control unit 46A of the robot 414 and acquires a patient's medical records and test results from an electronic medical record system. The analysis unit is implemented, for example, by a specific processing unit 290 of the data processing apparatus 12 and analyzes medical data using AI. The provision unit is implemented, for example, by the control unit 46A of the robot 414 and notifies the diagnostic result to a physician or patient. The remote support unit is implemented, for example, by the specific processing unit 290 of the data processing apparatus 12 and collects medical data from a remote medical institution and provides the analysis result. The rapid diagnosis unit is implemented, for example, by the control unit 46A of the robot 414 and promptly provides a diagnostic result to a patient who requires time to wait for diagnosis. The correspondence between each unit and the device or control unit is not limited to the above examples and various modifications are possible.

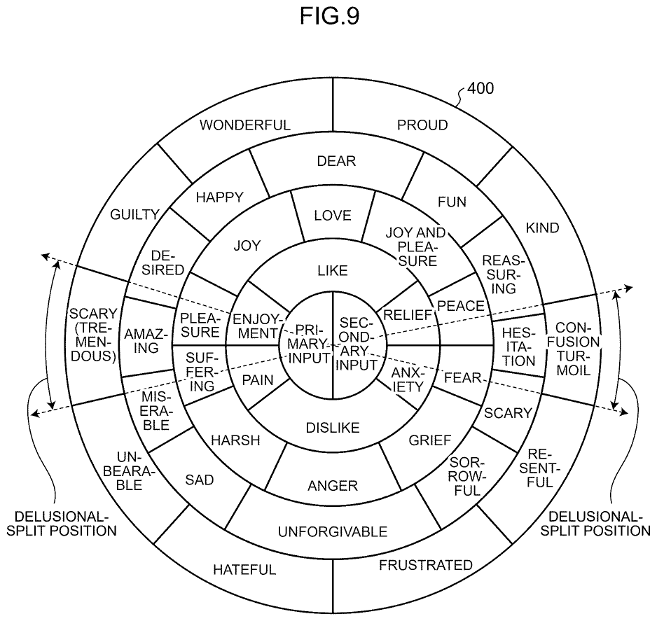

Note that the emotion identification model 59 as an emotion engine may determine the user's emotions according to a specific mapping. Specifically, the emotion identification model 59 may determine the user's emotions according to an emotion map, which is a specific mapping (see FIG. 9). Similarly, the emotion identification model 59 may determine the robot's emotions, and the specific processing unit 290 may perform specific processing using the robot's emotions.

FIG. 9 is a diagram showing an emotion map 400 where multiple emotions are mapped. In the emotion map 400, emotions are arranged concentrically radiating from the center. The closer to the center of the concentric circles, the more primitive the state of emotions is arranged. On the outer side of the concentric circles, emotions representing states and behaviors arising from mood are arranged. Emotions encompass concepts including emotional and mental states. On the left side of the concentric circles, emotions generally generated from reactions occurring in the brain are arranged. On the right side of the concentric circles, emotions generally induced by situational judgment are arranged. On the top and bottom of the concentric circles, emotions generated from reactions occurring in the brain and induced by situational judgment are arranged. Additionally, on the upper side of the concentric circles, “pleasant” emotions are arranged, and on the lower side, “unpleasant” emotions are arranged. In this way, in the emotion map 400, multiple emotions are mapped based on the structure from which emotions arise, and emotions that tend to occur simultaneously are mapped nearby.

These emotions are distributed in the 3 o'clock direction of the emotion map 400, and they usually move back and forth around reassurance and anxiety. In the right half of the emotion map 400, situational recognition takes precedence over internal sensations, giving a calm impression.

The inner side of the emotion map 400 represents the mind, and the outer side represents behavior, so the further out on the emotion map 400, the more visible (expressed in behavior) emotions become.

Here, human emotions are based on various balances like posture and blood sugar levels, and when these balances move away from the ideal, they indicate discomfort, and when they approach the ideal, they indicate comfort. In robots, cars, motorcycles, etc., emotions can be created based on various balances like posture and battery level, indicating discomfort when these balances move away from the ideal and comfort when they approach the ideal. The emotion map may be generated based on Dr. Mitsuyoshi's emotion map (Research on speech emotion recognition and brain physiological signal analysis systems related to emotions, Tokushima University, Doctoral dissertation: https://ci.nii.ac.jp/naid/500000375379). In the left half of the emotion map, emotions belonging to the domain called “reactions,” where sensations take precedence, are aligned. Additionally, in the right half of the emotion map, emotions belonging to the domain called “situations,” where situational recognition takes precedence, are aligned.

In the emotion map, two emotions that promote learning are defined. One is a negative emotion around “repentance” or “reflection” on the situation side. In other words, when a negative emotion arises in the robot, like “I never want to feel this way again” or “I don't want to be scolded again.” The other is an emotion around “desire” on the reaction side, which is positive. In other words, it is a positive feeling like “I want more” or “I want to know more.”

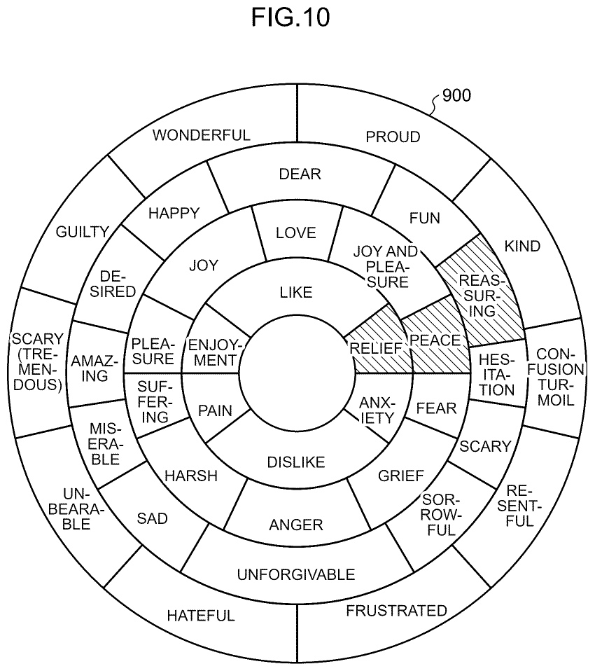

The emotion identification model 59 inputs user input into a pre-learned neural network, acquires emotion values indicating each emotion shown in the emotion map 400, and determines the user's emotions. This neural network is pre-learned based on multiple training data consisting of user input and combinations of emotion values indicating each emotion shown in the emotion map 400. Additionally, this neural network is learned so that emotions placed near each other in the emotion map 900 shown in FIG. 10 have similar values. FIG. 10 shows an example where multiple emotions like “reassured,” “calm,” and “confident” have similar emotion values.

In the above embodiments, an example form where specific processing is performed by a single computer 22 was described, but the technology disclosed herein is not limited to this, and distributed processing for specific processing by multiple computers including the computer 22 may be performed.

In the above embodiments, an example form where the specific processing program 56 is stored in the storage 32 was described, but the technology disclosed herein is not limited to this. For example, the specific processing program 56 may be stored in portable non-transitory storage media readable by a computer, such as a USB (Universal Serial Bus) memory. The specific processing program 56 stored in non-transitory storage media is installed in the computer 22 of the data processing device 12. The processor 28 executes specific processing according to the specific processing program 56.

Additionally, the specific processing program 56 may be stored in a storage device, such as a server connected to the data processing device 12 via the network 54, and downloaded and installed on the computer 22 in response to requests from the data processing device 12.

Furthermore, it is not necessary to store all of the specific processing program 56 in storage devices such as servers connected to the data processing device 12 via the network 54 or all in the storage 32, and a part of the specific processing program 56 may be stored.

Various processors, as shown next, can be used as hardware resources for executing specific processing. As processors, general-purpose processors that function as hardware resources for executing specific processing by executing software, i.e., programs, such as a CPU, can be mentioned. Additionally, as processors, dedicated electrical circuits with circuit configurations specially designed to execute specific processing, such as FPGA (Field-Programmable Gate Array), PLD (Programmable Logic Device), or ASIC (Application Specific Integrated Circuit), can be mentioned. Each processor has a built-in or connected memory, and each processor executes specific processing using the memory.

Hardware resources for executing specific processing may be composed of one of these various processors or a combination of two or more processors of the same or different types (e.g., a combination of multiple FPGAs or a combination of a CPU and FPGA). Additionally, hardware resources for executing specific processing may be a single processor.

As an example of composing with a single processor, firstly, there is a form where one or more CPUs and software are combined to constitute a single processor, which functions as hardware resources for executing specific processing. Secondly, there is a form using a processor, such as SoC (System-on-a-chip), that realizes the function of an entire system including multiple hardware resources for executing specific processing with a single IC chip. In this way, specific processing is realized using one or more of the various processors as hardware resources.

Furthermore, as a hardware structure of these various processors, more specifically, electrical circuits combined with circuit elements such as semiconductor elements can be used. Additionally, the specific processing described above is merely one example. Therefore, it goes without saying that unnecessary steps may be deleted, new steps may be added, or the order of processing may be changed within the scope not departing from the gist.

Additionally, in the examples described above, the explanation was divided into the first embodiment to the fourth embodiment, but parts or all of these embodiments may be combined. Additionally, the smart device 14, smart glasses 214, headset-type terminal 314, and robot 414 are examples, and each may be combined, or other devices may be used.

The descriptions and drawings shown above are detailed explanations of parts related to the technology disclosed herein and are merely examples of the technology disclosed herein. For example, the explanations regarding configurations, functions, actions, and effects above are explanations regarding examples of configurations, functions, actions, and effects of parts related to the technology disclosed herein. Therefore, it goes without saying that within the scope not departing from the gist of the technology disclosed herein, unnecessary parts may be deleted, new elements may be added, or replacements may be made to the descriptions and drawings shown above. Additionally, to avoid complexity and facilitate understanding of parts related to the technology disclosed herein, explanations concerning technical common knowledge and the like that do not require special explanation for enabling the implementation of the technology disclosed herein are omitted in the descriptions and drawings shown above.

All documents, patent applications, and technical standards described in this specification are incorporated by reference to the same extent as if each document, patent application, and technical standard were specifically and individually stated to be incorporated by reference in this specification.

(Supplementary Note 1) A system comprising: a collection unit configured to collect personal medical information; an analysis unit configured to analyze medical data collected by the collection unit; and a provision unit configured to provide a diagnostic result obtained by the analysis unit.

(Supplementary Note 2) The system according to Supplementary Note 1, further comprising a remote support unit configured to support use at a remote medical institution.

(Supplementary Note 3) The system according to Supplementary Note 1, further comprising a rapid diagnosis unit configured to promptly provide a diagnostic result to a patient who requires time to wait for diagnosis.

(Supplementary Note 4) The system according to Supplementary Note 1, wherein the collection unit is configured to collect data of a patient's medical records, test results, and medical images.

(Supplementary Note 5) The system according to Supplementary Note 1, wherein the analysis unit is configured to analyze medical data by AI and detect abnormalities.

(Supplementary Note 6) The system according to Supplementary Note 1, wherein the provision unit is configured to provide a diagnostic result analyzed by AI to a physician.

(Supplementary Note 7) The system according to Supplementary Note 1, wherein the collection unit is configured to estimate a patient's emotion and adjust the timing of medical data collection based on the estimated emotion of the patient.

(Supplementary Note 8) The system according to Supplementary Note 1, wherein the collection unit is configured to analyze a patient's past medical records and select a collection method.

(Supplementary Note 9) The system according to Supplementary Note 1, wherein the collection unit is configured to perform filtering based on a patient's current health condition and lifestyle habits when collecting medical data.

(Supplementary Note 10) The system according to Supplementary Note 1, wherein the collection unit is configured to estimate a patient's emotion and determine the priority of medical data to be collected based on the estimated emotion of the patient.

(Supplementary Note 11) The system according to Supplementary Note 1, wherein the collection unit is configured to consider a patient's geographic location information when collecting medical data and preferentially collect highly relevant data.

(Supplementary Note 12) The system according to Supplementary Note 1, wherein the collection unit is configured to analyze a patient's social media activity when collecting medical data and collect relevant data.

(Supplementary Note 13) The system according to Supplementary Note 1, wherein the analysis unit is configured to estimate a patient's emotion and adjust the expression method of analysis based on the estimated emotion of the patient.

(Supplementary Note 14) The system according to Supplementary Note 1, wherein the analysis unit is configured to adjust the level of detail of analysis based on the importance of medical data during analysis.

(Supplementary Note 15) The system according to Supplementary Note 1, wherein the analysis unit is configured to apply different analysis algorithms according to the category of medical data during analysis.

(Supplementary Note 16) The system according to Supplementary Note 1, wherein the analysis unit is configured to estimate a patient's emotion and adjust the length of analysis based on the estimated emotion of the patient.

(Supplementary Note 17) The system according to Supplementary Note 1, wherein the analysis unit is configured to determine the priority of analysis based on the submission timing of medical data during analysis.

(Supplementary Note 18) The system according to Supplementary Note 1, wherein the analysis unit is configured to adjust the order of analysis based on the relevance of medical data during analysis.

(Supplementary Note 19) The system according to Supplementary Note 1, wherein the provision unit is configured to estimate a patient's emotion and adjust the method of providing a diagnostic result based on the estimated emotion of the patient.

(Supplementary Note 20) The system according to Supplementary Note 1, wherein the provision unit is configured to adjust the level of detail of provision based on the importance of medical data when providing a diagnostic result.

(Supplementary Note 21) The system according to Supplementary Note 1, wherein the provision unit is configured to apply different provision algorithms according to the category of medical data when providing a diagnostic result.

(Supplementary Note 22) The system according to Supplementary Note 1, wherein the provision unit is configured to estimate a patient's emotion and adjust the length of a diagnostic result based on the estimated emotion of the patient.

(Supplementary Note 23) The system according to Supplementary Note 1, wherein the provision unit is configured to determine the priority of provision based on the submission timing of medical data when providing a diagnostic result.

(Supplementary Note 24) The system according to Supplementary Note 1, wherein the provision unit is configured to adjust the order of provision based on the relevance of medical data when providing a diagnostic result.

(Supplementary Note 25) The system according to Supplementary Note 2, wherein the remote support unit is configured to estimate a patient's emotion and adjust the method of remote support based on the estimated emotion of the patient.

(Supplementary Note 26) The system according to Supplementary Note 2, wherein the remote support unit is configured to estimate a patient's emotion and determine the priority of remote support based on the estimated emotion of the patient.

(Supplementary Note 27) The system according to Supplementary Note 2, wherein the remote support unit is configured to select a support method by considering a patient's geographic location information during remote support.

(Supplementary Note 28) The system according to Supplementary Note 3, wherein the rapid diagnosis unit is configured to estimate a patient's emotion and adjust the method of rapid diagnosis based on the estimated emotion of the patient.

(Supplementary Note 29) The system according to Supplementary Note 3, wherein the rapid diagnosis unit is configured to select a diagnostic method by referring to a patient's past medical records during rapid diagnosis.

(Supplementary Note 30) The system according to Supplementary Note 3, wherein the rapid diagnosis unit is configured to estimate a patient's emotion and determine the priority of rapid diagnosis based on the estimated emotion of the patient.

(Supplementary Note 31) The system according to Supplementary Note 3, wherein the rapid diagnosis unit is configured to select an optimal diagnostic method by considering a patient's geographic location information during rapid diagnosis.

What is claimed is:

1. A system comprising:
a communication interface configured to communicate with a client terminal and an external data source via a packet-switched network;
a processor;
a random-access memory;
a memory storing a data generation model obtained by deep learning on a neural network, and an emotion identification model;
a database; and
circuitry configured to:
receive, from the external data source via the communication interface and the packet-switched network, structured data comprising at least one of text data, numerical time-series data, or image data, and store the received structured data in the database;
analyze the structured data stored in the database by inputting the structured data into the data generation model to generate inference data comprising at least one of a probability score, a classification label, or a recommendation text;
estimate an emotion of a user by applying the emotion identification model to sensor data received from the client terminal via the communication interface; and
transmit the inference data to the client terminal via the communication interface and the packet-switched network, the inference data causing the client terminal to present the inference data to the user.

2. The system according to claim 1, wherein the structured data comprises medical data of a patient, the medical data comprising at least one of medical record text data, test result numerical vectors, or medical image tensors in DICOM format, and wherein the inference data comprises a diagnostic result comprising at least one of an abnormality probability score, a disease name label, or a recommended treatment policy text.

3. The system according to claim 2, wherein the data generation model comprises a plurality of AI models comprising at least one of a convolutional neural network configured to receive a medical image tensor and output a segmentation map of an abnormal region and an abnormality probability score, a recurrent neural network configured to receive a time-series array of test values and output a disease progression risk score, or a large language model configured to receive medical record text and output a disease name label and a recommended treatment policy text.

4. The system according to claim 2, wherein the circuitry is further configured to receive the medical data from an electronic medical record system of a medical institution via a secure communication protocol, assign a unique identifier to the medical data, and perform normalization and anonymization on the medical data before storing the medical data in the database.

5. The system according to claim 1, wherein the circuitry is further configured to adjust a timing of receiving the structured data from the external data source based on the estimated emotion, such that when the estimated emotion indicates anxiety, the circuitry delays the receiving, and when the estimated emotion indicates relaxation, the circuitry receives the structured data immediately.

6. The system according to claim 1, wherein the circuitry is further configured to analyze past data stored in the database associated with the user to select an optimal data collection method, the data collection method comprising at least one of a collection means or a collection frequency, the selection being performed by a machine learning algorithm comprising at least one of a decision tree, a random forest, or a reinforcement learning model.

7. The system according to claim 1, wherein the circuitry is further configured to receive, from the client terminal via the communication interface, attribute data of the user comprising at least one of a current health condition or a lifestyle habit, and to filter the structured data to be received from the external data source based on the attribute data by determining a priority score for each type of structured data.

8. The system according to claim 1, wherein the circuitry is further configured to determine a priority of the structured data to be received based on the estimated emotion, such that when the estimated emotion indicates anxiety, the circuitry prioritizes receiving structured data having a high urgency attribute, and when the estimated emotion indicates relaxation, the circuitry receives all structured data in a normal order.

9. The system according to claim 1, wherein the circuitry is further configured to receive geographic location information of the user from the client terminal via the communication interface, and to preferentially receive structured data associated with a geographic region corresponding to the geographic location information.

10. The system according to claim 1, wherein the circuitry is further configured to receive social media activity data of the user from the client terminal via the communication interface, analyze the social media activity data using a natural language processing model to extract status information of the user, and adjust a type of the structured data to be received from the external data source based on the extracted status information.

11. The system according to claim 1, wherein the circuitry is further configured to adjust an expression method of the inference data based on the estimated emotion, such that when the estimated emotion indicates anxiety, the circuitry generates the inference data in a simplified expression style, and when the estimated emotion indicates relaxation, the circuitry generates the inference data in a detailed expression style.

12. The system according to claim 1, wherein the circuitry is further configured to calculate an importance score for the structured data, and to adjust a level of detail of the analysis based on the importance score, such that for structured data having a high importance score, the circuitry applies a multi-stage analysis flow using a plurality of AI models, and for structured data having a low importance score, the circuitry applies a simplified analysis comprising threshold determination.

13. The system according to claim 1, wherein the circuitry is further configured to apply different analysis algorithms according to a category of the structured data, such that for image data, the circuitry applies a convolutional neural network, for numerical time-series data, the circuitry applies a recurrent neural network, and for text data, the circuitry applies a large language model.

14. The system according to claim 1, wherein the circuitry is further configured to adjust a length of the inference data based on the estimated emotion, such that when the estimated emotion indicates anxiety, the circuitry generates a concise inference result, and when the estimated emotion indicates relaxation, the circuitry generates a detailed inference result.

15. The system according to claim 1, wherein the circuitry is further configured to determine a priority of generating the inference data based on a submission timing associated with the structured data, such that structured data having a more recent submission timing is analyzed with a higher priority.

16. The system according to claim 1, wherein the circuitry is further configured to adjust a level of detail of the inference data to be transmitted based on an importance score associated with the structured data, such that for structured data having a high importance score, the circuitry generates detailed inference data, and for structured data having a low importance score, the circuitry generates a concise summary.

17. The system according to claim 1, wherein the circuitry is further configured to apply different inference data generation algorithms according to a category of the structured data when generating the inference data to be transmitted to the client terminal.

18. A system comprising:
a communication interface configured to communicate, via a packet-switched network conforming to at least one of a 5G, Wi-Fi, or Bluetooth communication standard, with a client terminal comprising a touch panel, a microphone, a speaker, a camera having a CMOS image sensor, and a display, and with an external data source;
a processor;
a random-access memory;
a memory storing a data generation model obtained by deep learning on a neural network, and an emotion identification model;
a database; and
circuitry configured to:
receive, from the external data source via the communication interface and the packet-switched network, structured data comprising at least one of text data, numerical time-series data, or image data, and store the received structured data in the database;
analyze the structured data stored in the database by inputting the structured data into the data generation model to generate inference data comprising at least one of a probability score, a classification label, or a recommendation text;
estimate an emotion of the user by applying the emotion identification model to at least one of voice data captured by the microphone or image data captured by the camera of the client terminal, received via the communication interface;
adjust at least one of a level of detail, an expression style, or a length of the inference data based on the estimated emotion; and
transmit the inference data to the client terminal via the communication interface and the packet-switched network, the inference data causing the client terminal to present the inference data to the user via at least one of the display or the speaker.

19. The system according to claim 18, wherein the data generation model comprises at least one of a text generation AI, an image generation AI, or a multimodal generation AI, and wherein the data generation model is a fine-tuned model configured to output inference results from prompts without instructions.

20. A method performed by circuitry of a data processing system comprising a processor, a random-access memory, a memory storing a data generation model obtained by deep learning on a neural network and an emotion identification model, a database, and a communication interface, the method comprising:
receiving, from an external data source via the communication interface and a packet-switched network, structured data comprising at least one of text data, numerical time-series data, or image data, and storing the received structured data in the database;
analyzing the structured data stored in the database by inputting the structured data into the data generation model to generate inference data comprising at least one of a probability score, a classification label, or a recommendation text;
estimating an emotion of a user by applying the emotion identification model to sensor data received from a client terminal via the communication interface; and
transmitting the inference data to the client terminal via the communication interface and the packet-switched network, the inference data causing the client terminal to present the inference data to the user.
